(19) United States
(12) Patent Application Publication
(10) Pub. No.: US 2023/0285327 A1
(43) Pub. Date: Sep. 14, 2023

(54) DESOXYSCALINE DERIVATIVES WITH MODIFIED MESCALINE-LIKE ACTION

(71) Applicant: Mind Medicine, Inc., New York, NY (US)

(72) Inventors: Daniel TRACHSEL, Detligen (CH); Matthias Emanuel LIECHTI, Oberwil (CH); Felix LUSTENBERGER, Meggen (CH)

(73) Assignee: Mind Medicine, Inc., New York, NY (US)

(21) Appl. No.: 18/196,992

(22) Filed: May 12, 2023

Related U.S. Application Data
(63) Continuation-in-part of application No. 17/987,922, filed on Nov. 16, 2022.
(60) Provisional application No. 63/280,439, filed on Nov. 17, 2021.

(51) Int. Cl. A61K 31/137

(52) U.S. Cl. CPC A61K 31/137

(57) ABSTRACT

A composition of a compound represented by FIG. 1 for use in substance-assisted therapy. A method of changing neurotransmission, by administering a pharmaceutically effective amount of a compound of FIG. 1 to a mammal, interacting with serotonin 5-HT2A receptors in the mammal, in particular also human beings, and inducing psychoactive effects. A method of treating a patient having adverse reactions to psychedelics by administering a desoxyscaline derivative to the patient, and avoiding adverse effects present with psychedelics. A method of changing neurotransmission of an individual, by administering a desoxyscaline derivative, and changing neurotransmission in the individual.

### BACKGROUND OF THE INVENTION

### 1. Technical Field

The present invention relates to both the substance definition and synthesis of desoxyscalines with modified mescaline-like action to be used in substance-assisted psychotherapy.

### 2. Background Art

Psychedelics are substances inducing unique subjective effects including dream-like alterations of consciousness, affective changes, enhanced introspective abilities, visual imagery, pseudo-hallucinations, synesthesia, mystical-type experiences, disembodiment, and ego-dissolution (Liechti, 2017; Passie et al., 2008).

Psychedelics, mainly lysergic acid diethylamide (LSD) and psilocin, are currently investigated as potential medications. First clinical trials indicate potential efficacy of LSD and psilocybin in addiction (Bogenschutz, 2013; Bogenschutz et al., 2015; Garcia-Romeu et al., 2019; Garcia-Romeu et al., 2015; Johnson et al., 2014; Johnson et al., 2016; Krebs et al., 2012), anxiety associated with life-threatening illness (Gasser et al., 2014; Gasser et al., 2015), depression (Carhart-Harris et al., 2021; Carhart-Harris et al., 2016a; Davis et al., 2021; Griffiths et al., 2016; Roseman et al., 2017; Ross et al., 2016), and anxiety (Griffiths et al., 2016; Grob et al., 2011; Ross et al., 2016). Several trials investigating therapeutic effects of LSD, psilocybin and other psychedelics are also ongoing, as can be seen on www.clinicaltrials.gov. There is also evidence that the psychedelic brew Ayahuasca which contains the active psychedelic substance N,N-dimethyltryptamine (DMT) (Dominguez-Clave et al., 2016) may alleviate depression (de Araujo, 2016; Dos Santos et al., 2016; Palhano-Fontes et al., 2019; Sanches et al., 2016). In contrast, there are no comparable therapeutic studies or elaborated concepts on the use of the psychedelic substance mescaline or related substances to treat medical conditions.

Although no psychedelic is currently licensed for medical use, psilocybin and LSD are used in special therapeutic-use programs (Schmid et al., 2021). Mescaline is a serotonergic psychedelic similar to LSD and psilocybin with comparable acute effects. Mescaline or its derivatives may be equally suitable to treat medical conditions. Specifically, existing psychedelic treatments such as LSD, psilocybin and DMT may not be suitable to be used in all patients considered for psychedelic-assisted therapy. The availability of several substances with different properties is important and the present lack thereof is a therapeutic problem which will further increase with more patients needing psychedelic-assisted therapy and an increase in demand for such treatment once the efficacy of first treatments will be documented in large clinical studies. For example, some patients may react with strong adverse responses to existing therapies such as psilocybin presenting with untoward effects including headaches, nausea/vomiting, anxiety, cardiovascular stimulation, or marked dysphoria. Thus, novel compounds with psychedelic-like action are needed.

Structurally, mescaline is a phenethylamine unlike LSD and psilocybin. LSD, psilocybin, and mescaline are all thought to induce their acute psychedelic effects primarily via their common stimulation of the 5-HT2A receptor. All serotonergic psychedelics including LSD, psilocybin, DMT, and mescaline are agonists at the 5-HT2A receptor (Rickli et al., 2016) and may therefore produce overall largely similar effects. However, there are differences in the receptor activation profiles and in the subsequent signal transduction pathway activation patterns between the substances that may induce different subjective effects. LSD potently stimulates the 5-HT2A receptor but also 5-HT2B/C, 5-HT1 and D1-3 receptors. Psilocin, i.e., the active metabolites present in the human body derived from the prodrug psilocybin, also stimulates the 5-HT2A receptor but additionally inhibits the 5-HT transporter (SERT). Mescaline binds in a similar, rather low concentration range to 5-HT2A, 5-HT2C, 5-HT1A and a2A receptors. In contrast to LSD, psilocybin and mescaline show no affinity for D2 receptors. Taken together, LSD can have greater dopaminergic activity than psilocybin and mescaline, psilocybin can have additional action at the SERT. Mescaline and its derivatives do not interact with the SERT in contrast to psilocybin. Taken together, the pharmacological profiles of LSD, psilocybin and mescaline show some differences, but it is not clear whether these are reflected by differences in their psychoactive profiles in humans. This is currently being investigated in a clinical study (www.clinicaltrials.gov: NCT04227756).

In humans, subjective effects or psychoactive doses of mescaline appear within 30 minutes, peak at 4 hours and dose-dependently last 10-16 hours. The plasma half-life is approximately 6 hours (Charalampous, 1966).

The acute subjective effects of psychedelics are mostly positive in most humans (Carhart-Harris et al., 2016b; Dolder et al., 2016; Dolder et al., 2017; Holze et al., 2019; Schmid et al., 2015). However, there are also negative subjective effects such as anxiety in many humans likely depending on the dose used, personality traits (set), the setting (physical and social environment) and other factors. The induction of an overall positive acute response to the psychedelic is critical because several studies showed that a more positive experience is predictive of a greater therapeutic long-term effect of the psychedelic (Garcia-Romeu et al., 2015; Griffiths et al., 2016; Ross et al., 2016). Even in healthy subjects, a more positive acute response to a psychedelic including LSD has been shown to be linked to more positive long-term effects on well-being (Griffiths et al., 2008; Schmid et al., 2018).

Mescaline has relevant acute side effects to different degrees depending on the subject treated and including increased blood pressure, nausea and vomiting, negative body sensations, and dysphoria. Such side effects of a substance are often linked to its interactions with pharmacological targets. For example, interactions with adrenergic receptors may result untoward clinical cardio-stimulant properties. Additionally, changes in the relative activation profile of serotonin 5-HT receptors and other targets change the quality of the psychoactive effects. Alterations in the binding potency, the binding mode, and the potency in activating the subsequent signaling pathways at 5-HT2A receptors as well as the molecule's lipophilicity may mostly determine the clinical dose to induce psychoactive effects. Alterations changing the metabolic stability of the compounds can also change the duration of action of the substance significantly.

New mescaline-based derivatives are needed to provide substances with an improved effect profile such as, but not limited to, more positive effects, less adverse effects, different qualitative effects, and shorter or longer duration of acute effect.

### SUMMARY OF THE INVENTION

The present invention provides for a composition of a compound represented by FIG. 1 for use in substance-assisted therapy, wherein:

Rₐₗₚₕₐ₁ and Rₐₗₚₕₐ₂ are, independently, hydrogen, deuteron, methyl, ethyl, deuterated methyl (D₁-D₃), or deuterated ethyl (D₁-D₅), and

R′ is

C₁-C₅ branched or unbranched alkyl with the alkyl optionally substituted with F₁-F₁₁ fluorine and/or D₁-D₁₁ deuteron substituents up to a fully fluorinated and/or deuterated alkyl,

C₃-C₆ cycloalkyl optionally and independently substituted with one or more substituents such as F₁-F₁₅ fluorine and/or D₁-D₁₅ deuteron and/or C₁-C₂ alkyl,

(C₃-C₆ cycloalkyl)-C₁-C₂ branched or unbranched alkyl optionally substituted with one or more substituents such as F₁-F₁₅ fluorine and/or D₁-D₁₅ deuteron and/or C₁-C₂ alkyl,

C₂-C₅ branched or unbranched alkenyl with E or Z or cis or trans double bond configuration, where any of the carbons of the branched or unbranched alkenyl substituent is optionally substituted independently and in any combination with one or more C₁-C₂ alkyl, with F₁-F₁₃ fluorine, with D₁-D₁₃ deuteron, with C₂ alkenyl or with aryl or heteroaryl bearing no up to any number of ether, thioether, halogen, alkyl, fluorinated alkyl, alkenyl, alkynyl or nitrogen-containing substituents,

C₂-C₅ branched or unbranched alkynyl where any of the carbons of the branched or unbranched alkynyl substituent is optionally substituted independently and in any combination with one or more C₁-C₂ alkyl, with F₁-F₁₁ fluorine, with D₁-D₁₁ deuteron, with C₂ alkenyl or with aryl or heteroaryl bearing no up to any number of ether, thioether, halogen, alkyl, fluorinated alkyl, alkenyl, alkynyl or nitrogen-containing substituents,

any halogen or

a nitrogen-containing substituent such as CN or NO₂.

The present invention provides a method of changing neurotransmission, by administering a pharmaceutically effective amount of a compound of FIG. 1 to a mammal, interacting with serotonin 5-HT2A receptors in the mammal, in particular also human beings, and inducing psychoactive effects.

The present invention also provides for a method of treating a patient having adverse reactions to psychedelics by administering a desoxyscaline derivative to the patient, and avoiding adverse effects present with psychedelics.

The present invention also provides for a method of changing neurotransmission of an individual, by administering a desoxyscaline derivative, and changing neurotransmission in the individual.

### DESCRIPTION OF THE DRAWINGS

Other advantages of the present invention are readily appreciated as the same becomes better understood by reference to the following detailed description when considered in connection with the accompanying drawings wherein:

FIG. 1 shows the chemical structure of desoxyscalines where Rₐₗₚₕₐ₁ and Rₐₗₚₕₐ₂ are, independently, hydrogen, deuteron, methyl, ethyl, deuterated methyl (D₁-D₃), or deuterated ethyl (D₁-D₅); R′ is 1.) C₁-C₅ branched or unbranched alkyl with the alkyl optionally substituted with F₁-F₁₁ fluorine and/or D₁-D₁₁ deuteron substituents up to a fully fluorinated and/or deuterated alkyl, 2.) C₃-C₆ cycloalkyl optionally and independently substituted with one or more substituents such as F₁-F₁₅ fluorine and/or D₁-D₁₅ deuteron and/or C₁-C₂ alkyl, 3.) (C₃-C₆ cycloalkyl)-C₁-C₂ branched or unbranched alkyl optionally substituted with one or more substituents such as F₁-F₁₅ fluorine and/or D₁-D₁₅ deuteron and/or C₁-C₂ alkyl, 4.) C₂-C₅ branched or unbranched alkenyl with E or Z or cis or trans double bond configuration, where any of the carbons of the branched or unbranched alkenyl substituent is optionally substituted independently and in any combination with one or more C₁-C₂ alkyl, with F₁-F₁₃ fluorine, with D₁-D₁₃ deuteron, with C₂ alkenyl or with aryl or heteroaryl bearing no up to any number of ether, thioether, halogen, alkyl, fluorinated alkyl, alkenyl, alkynyl or nitrogen-containing substituents, 5.) C₂-C₅ branched or unbranched alkynyl where any of the carbons of the branched or unbranched alkynyl substituent is optionally substituted independently and in any combination with one or more C₁-C₂ alkyl, with F₁-F₁₁ fluorine, with D₁-D₁₁ deuteron, with C₂ alkenyl or with aryl or heteroaryl bearing no up to any number of ether, thioether, halogen, alkyl, fluorinated alkyl, alkenyl, alkynyl or nitrogen-containing substituents, 6.) any halogen or 7.) a nitrogen-containing substituent such as CN or NO₂;

FIG. 2 exhibits examples (compounds 6a-6c and 7a-7d) of desoxyscaline derivatives represented by FIG. 1 within the scope of the invention;

FIG. 3 exhibits examples (compounds 11-12, 19a-19b and 20a-20f) of desoxyscaline derivatives represented by FIG. 1 within the scope of the invention;

FIG. 4 exhibits examples (compounds 22a-22c and 30-33) of desoxyscaline derivatives represented by FIG. 1 within the scope of the invention;

FIG. 5 summarily describes the synthetic route to the desoxyscalines derivatives compounds 6a-6c and 7a-7d via a “classic” route (preparation of the corresponding aldehydes, converting them to the nitroolefins and then reducing them to the desired final compounds);

FIG. 6 summarily describes the synthetic route to the desoxyscalines 11-12, as well as 19a-19b and 20a-20f via the synthesis of appropriate templates (compounds 15 and 16), which are used for parallel diversification, namely in a Wittig reaction;

FIG. 7 summarily describes the synthetic route to the desoxyscalines 22a-22c via the use of an appropriate template (compound 13), which is used for parallel diversification, namely in a Stille reaction;

FIG. 8 summarily describes the synthetic route to the fluorinated desoxyscalines 30-33; and

FIG. 9 is a summary of binding properties with 5HT2A and 5HT2B receptors of compounds of the present invention.

### DETAILED DESCRIPTION OF THE INVENTION

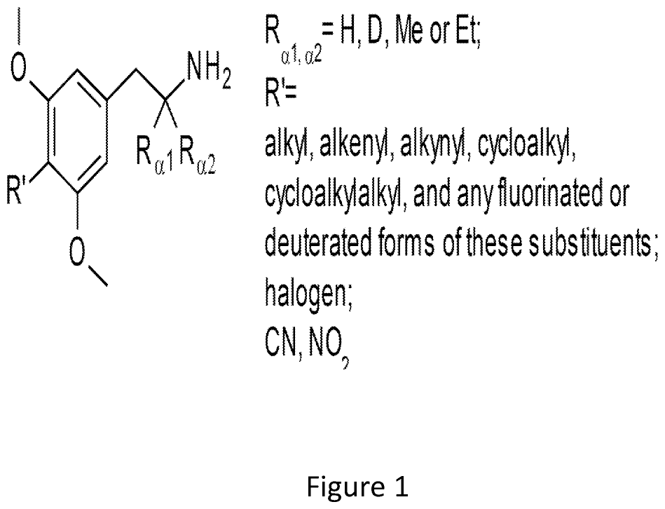

The present invention provides for desoxyscaline derivatives. More specifically, the present invention provides for a composition of a compound represented by FIG. 1 for use in substance-assisted therapy, wherein:

Rₐₗₚₕₐ₁ and Rₐₗₚₕₐ₂ are, independently, hydrogen, deuteron, methyl, ethyl, deuterated methyl (D₁-D₃), or deuterated ethyl (D₁-D₅), and

R′ is

C₁-C₅ branched or unbranched alkyl with the alkyl optionally substituted with F₁-F₁₁ fluorine and/or D₁-D₁₁ deuteron substituents up to a fully fluorinated and/or deuterated alkyl,

C₃-C₆ cycloalkyl optionally and independently substituted with one or more substituents such as F₁-F₁₅ fluorine and/or D₁-D₁₅ deuteron and/or C₁-C₂ alkyl,

(C₃-C₆ cycloalkyl)-C₁-C₂ branched or unbranched alkyl optionally substituted with one or more substituents such as F₁-F₁₅ fluorine and/or D₁-D₁₅ deuteron and/or C₁-C₂ alkyl,

C₂-C₅ branched or unbranched alkenyl with E or Z or cis or trans double bond configuration, where any of the carbons of the branched or unbranched alkenyl substituent is optionally substituted independently and in any combination with one or more C₁-C₂ alkyl, with F₁-F₁₃ fluorine, with D₁-D₁₃ deuteron, with C₂ alkenyl or with aryl or heteroaryl bearing no up to any number of ether, thioether, halogen, alkyl, fluorinated alkyl, alkenyl, alkynyl or nitrogen-containing substituents,

C₂-C₅ branched or unbranched alkynyl where any of the carbons of the branched or unbranched alkynyl substituent is optionally substituted independently and in any combination with one or more C₁-C₂ alkyl, with F₁-F₁₁ fluorine, with D₁-D₁₁ deuteron, with C₂ alkenyl or with aryl or heteroaryl bearing no up to any number of ether, thioether, halogen, alkyl, fluorinated alkyl, alkenyl, alkynyl or nitrogen-containing substituents,

any halogen or

a nitrogen-containing substituent such as CN or NO₂.

In addition to the aforementioned description of compounds represented by FIG. 1, any non-protic hydrogen can be replaced by a deuteron. Specifically, this complements to compounds represented by FIG. 1 by bearing one deuteron up to a completely deuterated compound and any stereoisomers thereof. More specifically, a deuteration can be conducted, independently and in any combination, on any of the methoxy groups, on the aromatic nucleus, or on the alkylamine sidechain.

Rₐₗₚₕₐ₁ and Rₐₗₚₕₐ₂ beyond C2, i.e., longer than methyl or ethyl, are not recommended as they result in compounds that are much less or even completely not pharmacologically active at the target site (5-HT2A receptor).

The compounds represented by FIG. 1 are basic compounds which form acid addition salts with inorganic or organic acids. Therefore, they form pharmaceutically acceptable inorganic and organic salts with pharmacologically acceptable inorganic or organic acids. Acids to form such salts can be selected from inorganic acids such as hydrochloric acid, hydrobromic acid, hydroiodic acid, sulfuric acid, nitric acid, phosphoric acid, and the like, and organic acids, such as carbonic acid, p-toluene-sulfonic acid, methane-sulfonic acid, oxalic acid, succinic acid, citric acid, benzoic acid, and the like. Examples of such pharmaceutically acceptable salts thus are sulfate, pyrosulfate, bisulfate, sulfite, bisulfite, phosphate, monohydrogen-phosphate, dihydrogen-phosphate, metaphosphate, pyro-phosphate, chloride, bromide, iodide, formate, acetate, propionate, decanoate, caprylate, acrylate, isobutyrate, caproate, heptanoate, oxalate, malonate, succinate, suberate, sebacate, fumarate, maleate, benzoate, phthalate, sulfonate, phenylacetate, citrate, lactate, glycollate, tartrate, methane sulfonate, propane sulfonate, mandelate and the like. Preferred pharmaceutically acceptable salts are those formed with hydrochloric acid.

Furthermore, and without loss of generality and elaborating on details, the invention includes any prodrugs, i.e., any chemical modification of the described compounds that is (metabolically) converted to the described compound in the human body (circulation).

The general chemical terms used for FIG. 1 have their usual meanings. For example, the term “alkyl” includes such groups as methyl, ethyl, n-propyl, isopropyl, n-butyl, isobutyl, sec-butyl, tert-butyl, and the like. For another example, the term “cycloalkyl” includes such groups as cyclopropyl, cyclobutyl, cyclopentyl, and the like. Further on, the term “alkenyl” includes such groups as vinyl (ethenyl), 1-propenyl, 2-propenyl, isopropenyl, butenyl, and the like. The term “alkynyl” includes groups such as ethynyl, 1-propynyl, 2-propynyl, 1-butynyl, 2-butynyl, 3-butynyl, phenyl ethynyl, and the like. The term “halogen” includes fluorine, chlorine, bromine, and iodine substituent.

Those skilled in the art will appreciate that certain of the compounds of the present invention have at least one chiral carbon, and can therefore exist as a racemate, as individual enantiomers or diastereomers, and as mixtures of individual enantiomers or diastereomers in any ratio. For example, individual enantiomers of compounds of the invention are illustrated in FIG. 1 where Rₐₗₚₕₐ₁ is different from Rₐₗₚₕₐ₂. Those skilled in the art will also appreciate that those compounds of the invention where R′ in FIG. 1 consists of a chiral substituent, can bear an additional asymmetric center which create additional optical isomers as described above. While it is a preferred embodiment of the invention that the compounds of the invention exist are used as racemates or mixtures of diastereomers, the present invention also contemplates the compounds of the invention existing in individual enantiomeric or diastereomeric pure form.

Those skilled in the art will also appreciate that certain of the compounds of the present invention have at least one double bond leading, depending on the double bond's substituents, to cis/trans or E/Z configurational isomerism. While it is a preferred embodiment of the invention that the compounds of the invention exist are used as pure configurational isomers, the present invention also contemplates the compounds of the invention existing in individual cis/trans or E/Z mixtures, respectively.

The individual enantiomers and diastereomers can be prepared by chiral chromatography of the racemic or enantiomerically or diastereomerically enriched amine, or fractional crystallization of salts prepared from racemic- or enantiomerically- or diastereomerically-enriched amine and a chiral acid. Alternatively, the free amine can be reacted with a chiral auxiliary and the enantiomers or diastereomers separated by chromatography or crystallization followed by removal of the chiral auxiliary to regenerate the free amine. Furthermore, separation of enantiomers or diastereomers can be performed at any convenient point in the synthesis of the compounds of the invention. The compounds of the invention can also be prepared by application of chiral syntheses. The compound itself is a pharmacologically acceptable acid addition salt thereof.

The individual cis/trans or E/Z configurational isomers can be accessed by either selective synthesis or by separation techniques addressing the different physicochemical properties of the configurational isomers by applying techniques such as chromatography, crystallization, distillation, or extraction.

In patients that have adverse reactions to other psychedelics, desoxyscaline derivatives can be useful as alternative treatments. In some patients, desoxyscalines can also be useful because another experience than made with mescaline, psilocybin or LSD is necessary or because a patient is not suited for therapy with these existing approaches a priori. Thus, desoxyscaline derivatives of FIG. 1 can serve as alternative treatment options with characteristics sufficiently similar to other psychedelics to be therapeutic but also sufficiently different to provide added benefits or avoid negative effects of other psychedelics.

Based on structural relations, the compounds of FIG. 1 described in the present invention are expected to have overall similar pharmacological properties as mescaline or other phenethylamine-based psychedelics as described above.

This assumption is further emphasized by the only known and described desoxyscaline, a compound named “DESOXY”, which has shown psychoactive effects in human (Shulgin et al., 1991).

The present invention provides compounds of FIG. 1 that are pharmacologically active and allow changing the neurotransmission and/or producing neurogenesis. More specifically, but not excluding, the compounds interact with serotonin (5-HT, 5-hydroxytryptamine) 5-HT2A and 5-HT2C receptors in mammals by administering to a mammal in need of such interaction a pharmaceutically effective amount of a compound of FIG. 1.

Therefore, the present invention provides a method of changing neurotransmission, by administering a pharmaceutically effective amount of a compound of FIG. 1 to a mammal, increasing serotonin 5-HT2A receptor interaction in the mammal, and inducing psychoactive effects.

The neuronal interaction of compounds represented in FIG. 1 can be used in mammals for substance-assisted psychotherapy where the compounds induce psychoactive effect to enhance psychotherapy. The preferred mammal is human.

The intensity and quality of the psychoactive effect including psychedelic or empathogenic effects, the quality of perceptual alterations such as imagery, fantasy and closed or open eyes visuals, and body sensation changes, the pharmacologically active doses, can be different or similar to that of mescaline.

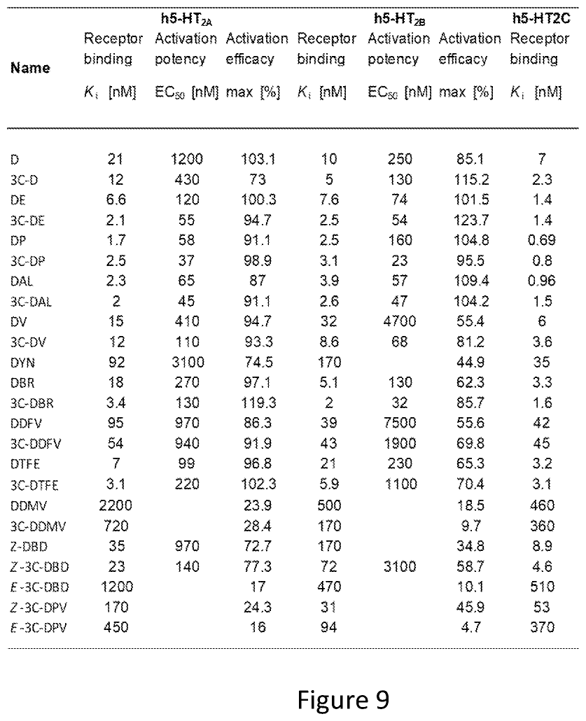

Mescaline and its 4-alkoxy homologs as well as its amphetamine counterparts are known to interact with serotonin 5-HT2A receptors (Kolaczynska, LUthi, Trachsel, Hoener, Liechti, FIG. 9).

Some of the invented desoxyscaline compounds represented by FIG. 1 have shown markedly increased affinities at the aforementioned receptors (Trachsel, Hoener, Liechti, FIG. 9) in comparison to mescaline and indicating psychedelic action.

Not only receptor interactions can change by structural modifications represented in FIG. 1 but also the metabolism can be modified significantly by making, as an example, but is not limited to, a rather labile vinyl or ethynyl compound more or less prone to metabolism by introducing alkyl groups, aryl or heteroaryl groups, fluorine atoms and deuterium atoms to this functional group in either vinyl, allyl or gamma positions, or in ethynyl or propargyl positions as aforementioned. Thus, the invention allows also for the synthesis of psychedelic compounds with a relatively shorter duration of action compared to more metabolically stable and longer-acting compounds.

The structure of 4-oxygen-free mescaline derivatives represented in FIG. 1 was previously only described for one single compound (Benington et al., 1960; Shulgin et al., 1991), namely the compound 4-desoxymescaline (3,5-dimethoxy-4-methylphenethylamine). Only some behavioral data in cat and curtly acute effect including duration of action and dose (Shulgin et al., 1991) was mentioned, not the pharmacological profiles and human therapeutic uses.

Desoxyscaline derivatives can include 4-oxygen-free substitution variations of the phenethylamine structure forming “desoxyscalines” or can include the addition of the methylation of the alpha carbon of the phenethylamine structure to form amphetamines also containing the above 3,4,5-substitutions on the phenyl ring to form “3C-desoxyscalines” (Shulgin et al., 1991; Trachsel et al., 2013). Further on, the aforementioned alpha carbon can also be substituted, independently and in any combination, with one or two deuterons, methyl, ethyl, deuterated methyl (D₁-D₃), or deuterated ethyl (D₁-D₅) groups. Several new such desoxyscaline derivatives represented in FIG. 1 were newly synthesized in the present invention.

While all the desoxyscaline derivatives represented in FIG. 1 are useful in optimizing the clinical effect profile of psychedelics, certain classes of the compounds are preferred, such as wherein the compound is a free base, a salt, a hydrochloride salt, a racemate where applicable, a single enantiomer, a single diastereomer, or a mixture of enantiomers or diastereomers in any ratio, or an individual of a cis/trans or E/Z configurational isomer, or a mixture of these configurational isomers in any ratio. It will be understood that these classes can be combined to form additional preferred classes.

The synthetic access to 4-substituted 3,5-dimethoxyphenethylamines and their amphetamine counterparts is rather challenging, except for 4-alkoxy derivatives (4-homoscalines) that have been described thoroughly by, e.g., the inventors earlier (provisional patent application entitled: “mescaline derivatives with modified action”, U.S. 63/153,317, filing date 2/24/2021). A general strategy to access some of the compounds of the field of invention has been set up as follows. The direct introduction of the 4-substituent could be achieved by using the ipso substitution method described by Azzena et al. (Azzena et al., 1990). Following this, 3,4,5-trimethoxybenzaldehyde dimethyl acetal was treated with elemental sodium in THF anhydr. For a prolonged time and then the intermediate was allowed to react with an alkyl halide. Acidic hydrolysis of the carbonyl protected intermediate afforded the corresponding benzaldehyde. Another route of access of such benzaldehydes was described by Comins et al. (Comins et al., 1984), wherein an in situ carbonyl protection of the 4-unsubstituted 3,5-dimethoxybenzaldehyde and subsequent ortho-lithiation was applied for introduction of a 4-alkyl substituent. The preparation of the nitroolefins from these 4-alkylated benzaldehydes was achieved by the reaction with nitromethane or nitroethane, generally referred as the Henry reaction, using n-butylamine and acetic acid as well as molecular sieves, an advantageous catalytic system described earlier by the inventors (provisional patent application entitled: “mescaline derivatives with modified action”, U.S. 63/153,317, filing date 2/24/2021). The nitroolefins are reduced to the corresponding desoxyscalines or 3C-desoxyscalines by using lithium aluminum hydride (LAH) or alane generated in situ from LAH and concentrated sulfuric acid, in analogy to earlier works (Shulgin et al., 1991; Trachsel, 2002).

However, the synthetic access invented and described before is not always suitable for accessing compounds represented in FIG. 1 containing a further functionalized 4-substituent. Thus, additional routes were set up, and a parallel synthesis approach that allows a structural diversification late in the synthesis was chosen, reducing synthetic efforts. Following this, N-trifluoroacetyl protected 4-bromo-3,5-dimethoxyphenethylamine as well as N-trifluoroacetyl protected 4-bromo-3,5-dimethoxyamphetamine were prepared as valuable building blocks. They could be converted to 1.) the corresponding 4-formyl derivatives which were then allowed to react with Wittig ylides to form 4-aryl-conjugated alkenes, or 2.) the corresponding 4-aryl alkylated, alkenylated or alkynated compounds by applying C—C Stille coupling reactions. Any of the N-trifluoroacetyl protected products accessed by the routes described before could then be N-deprotected in order to access final compounds represented in FIG. 1.

Any of the unsaturated 4-substituent introduced as described before can also be reduced by classical conditions such as the use of hydrogen and a catalyst such as palladium on activated charcoal to access the corresponding 4-alkylated derivatives.

4-fluorinated vinyl or 4-fluorinated alkyl substituted derivatives are yet another type of compounds invented and represented by FIG. 1 and can also be accessed, as an illustrative example, by allowing to react a Wittig-type internal salt ((triphenylphosphonio)difluoroacetate (Zheng et al., 2013)) with suitable 4-formylated intermediates and subsequent reaction with tetrabutylammonium fluoride.

The group presented in the preparation section, namely compounds 6a to 6c, 7a to 7d, 11 to 12, 19a to 19b, 20a to 20f, 22a to 22c and 30 to 33, is illustrative of desoxyscaline derivatives represented in FIG. 1 contemplated within the scope of the invention.

A small selection of the synthesized desoxyscalines and their amphetamine congeners were investigated at the key target for psychoactive effects in vitro (Trachsel, Hoener, Liechti, FIG. 9). The main target of psychedelics is the 5-HT2A receptor (Holze et al., 2021) and typically there is a high affinity binding at this receptor (Rickli et al., 2016). Additionally, the binding potency at the 5-HT2A receptor is typically predictive of the human doses of psychedelics to be psychoactive for many compounds (Luethi et al., 2018). Furthermore, the psychedelic effects of psilocybin in humans have been shown to correlate with 5-HT2A receptor occupancy measures using positron emission tomography (Madsen et al., 2019). Thus, interactions with this target are relevant and predict psychedelic action with high likelihood for most psychedelics. However, this may not be the case for all substances within this class.

Additional receptors such as the serotonergic 5-HT1A and 5-HT2C or dopaminergic D2 receptors are thought to moderate the effects of psychedelics (Rickli et al., 2016). Although some psychedelics like psilocybin do not directly act on dopaminergic receptors, they have nevertheless some dopaminergic properties by releasing dopamine in the striatum (Vollenweider et al., 1999) likely via 5-HT1A receptor activation (Ichikawa et al., 2000). Furthermore, LSD has activity at D2 receptors (Rickli et al., 2016) and some of its behavioral effect may be linked to this target (Marona-Lewicka et al., 2005).

Activity of compounds at monoamine transporters are thought to mediate MDMA-like empathogenic effects (Hysek et al., 2012). Importantly, mescaline is a very weak 5-HT2A receptor ligand and high doses are needed to induce psychoactive effects in humans. However, despite its low potency, mescaline can have extraordinarily strong psychedelic effects in humans at high doses and the same is likely the case for the substances developed within the present invention although 10× to 20× higher potency is also possible in some desoxyscaline compounds, to be evaluated in detail clinically. Key results of the preliminary pharmacological profiling of the compounds described herein were:

Some of the desoxyscaline derivatives represented in FIG. 1 showed relevant binding affinity at the serotonin 5-HT2A receptor indicating activity as psychedelics. For some compounds, the binding affinity was highly superior to mescaline.

Together, the in vitro profiles of mescaline and its desoxy derivatives represented in FIG. 1 compared with that of psilocin and LSD indicate overall psychedelic properties when used in humans. Accordingly, some desoxyscaline derivatives can exert psychedelic acute effect profiles that are more beneficial to some patients including but not limited to: more overall positive effects, more or less perceptual effects, more emotional effects, less anxiety, less cardio-stimulant effects, less adverse effects, less nausea, longer and also shorter effects among other properties and compared to mescaline.

There are several problems when using mescaline that can be solved using the compounds described herein. Namely, high doses of mescaline (200-800 mg) are needed to induce a full psychedelic experience. Derivatives represented in FIG. 1 can be more potent resulting in reduced need of the substance. Psychedelics like psilocybin produce adverse effects including nausea and vomiting, cardiovascular stimulation, and an increase in body temperature and others. The novel compounds can produce less nausea, less cardio stimulation, less thermogenesis and/or other adverse responses. Mescaline has a long duration of action. The presently developed substances were designed to have similar qualitative effects to mescaline while acting shorter or to have a long duration of action but other qualitative effects as reflected by their structural changes and associated pharmacological properties. In particular, metabolically less-stable compounds were created to shorten the plasma half-life and duration of action in humans. Other alterations of the chemical structure were designed to create substances with qualitative effects different from those of mescaline and creating subjective effects that are considered beneficial to assist psychotherapy including feelings of empathy, openness, trust, insight, and connectedness and known to those knowledgeable in the field.

The compounds represented by FIG. 1 act with shorter, with similar, or with longer duration of action in human in comparison to the original mescaline molecule. This is triggered by modification of the molecular structure in FIG. 1.

The present invention therefore also provides for a method of treating a patient having adverse reactions to psychedelics by administering a desoxyscaline derivative to the patient, and avoiding adverse effects present with psychedelics.

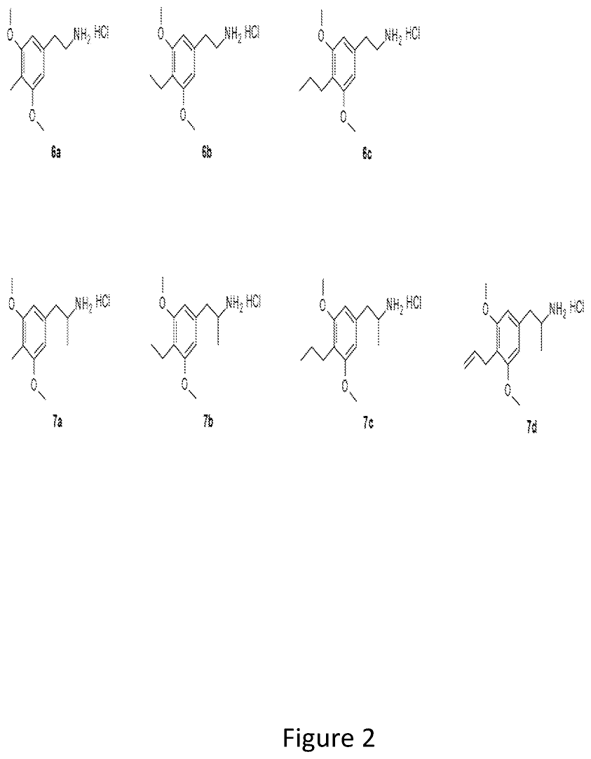

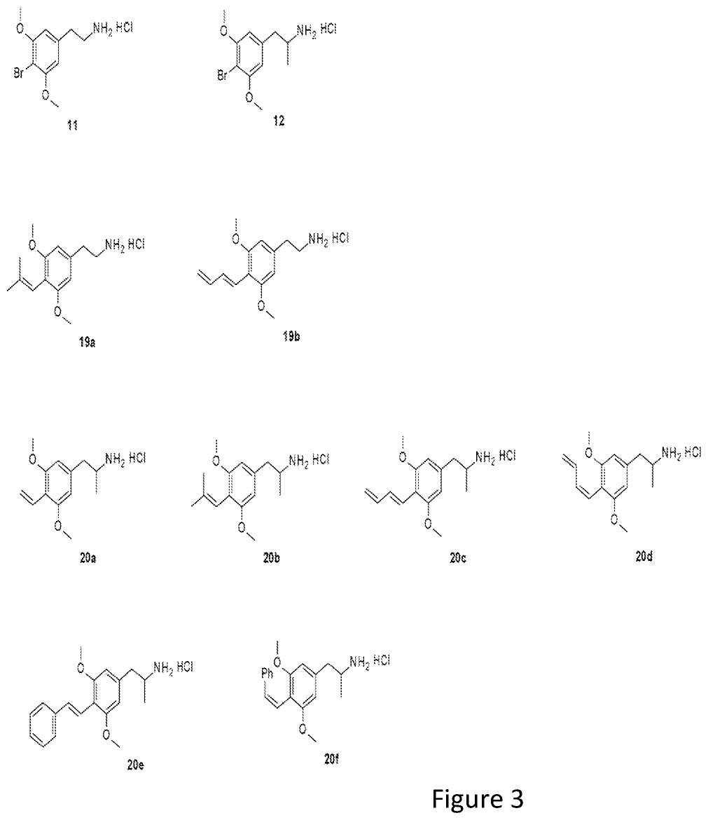

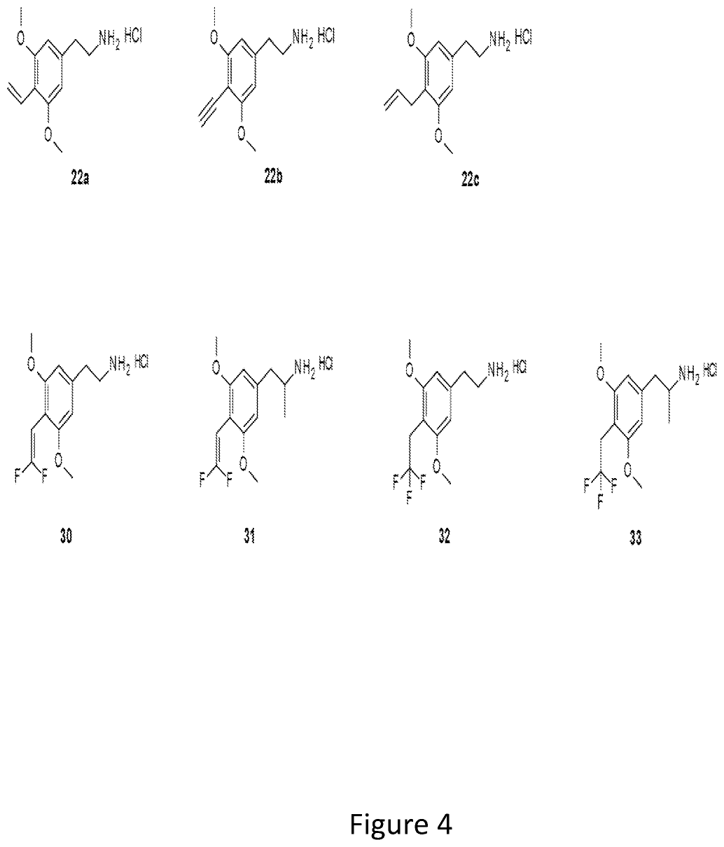

The group presented in the preparation section, namely compounds 6a to 6c, 7a to 7d, 11 to 12, 19a to 19b, 20a to 20f, 22a to 22c and 30 to 33 (chemical structures see FIGS. 2-4), is illustrative of desoxyscaline derivatives represented in FIG. 1 contemplated within the scope of the invention.

The compounds according to the invention and represented in FIG. 1 allow modification of the mode of action, the psychodynamic processes, and the qualitative perceptions, e.g., in terms of psychedelic or empathogenic intensity in comparison to the original mescaline molecule.

The compounds, according to the invention and represented in FIG. 1 can cause similar or different quality of imagery, fantasy and closed or open eyes visuals in comparison to the original mescaline molecule.

The compounds, according to the invention and represented in FIG. 1 can have a similar or a higher dose potency in comparison to the original mescaline molecule.

The compounds, according to the invention and represented in FIG. 1 can cause similar or more favorable body feelings in comparison to the original mescaline molecule.

The aforementioned characteristics can be modified in a progressive way by the introduction of one or more fluorine atoms, by one or more deuterium atoms and by one or more alkyl groups, independently as well as in any combination, to the 4-alkyl, 4-cycloalkyl, 4-cycloalkylalkyl, 4-alkenyl or 4-alkynyl group in any position of these substituents (position 4 represented as “R” in FIG. 1).

The modified properties can be tailored and applied individually to the patient's need. This is not only targeted by changing the compound's receptor profile but also greatly by the modification of ADME (Absorption, Distribution, Metabolism and Excretion) via the introduction of more, similar or less liable 4-substituents “R” in compounds represented in FIG. 1.

### Preparation of the Compounds

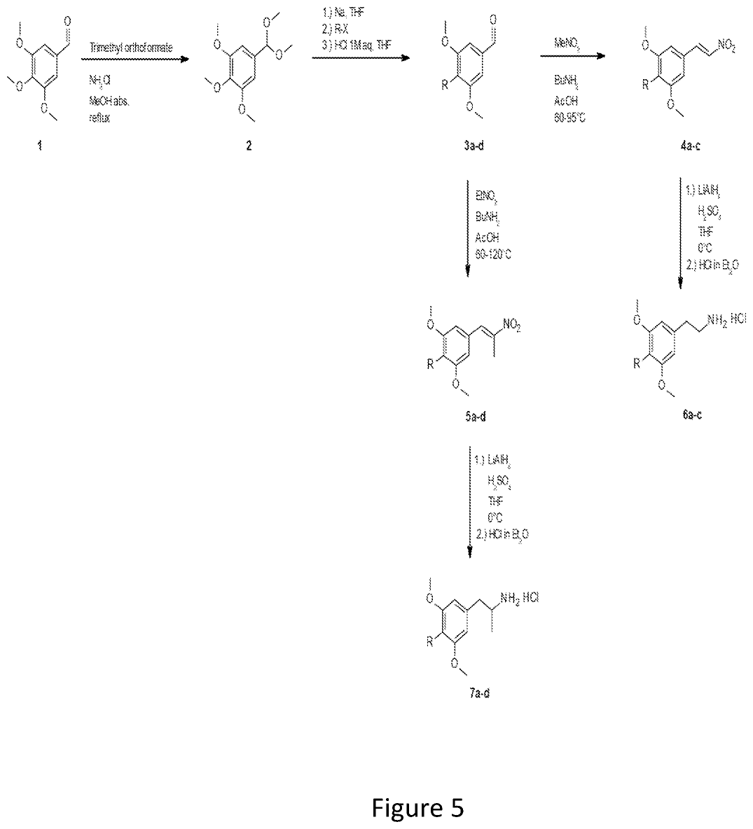

A general access to the desoxyscalines and 3C-desoxyscalines is outlined in FIGS. 5 to 8. Commercially available 3,4,5-trimethoxybenzaldehyde (1) is carbonyl-protected to the dimethyl acetal 2 (FIG. 5). Next, 2 is allowed to react with elemental sodium in a suitable organic solvent such as tetrahydrofuran (THF) anhydr. And then trapped with an alkyl halide. The corresponding 4-alkylated acetals are then deprotected to get the corresponding 4-alkylated aldehydes (such as illustrated in FIG. 5, compounds 3a-d) by using an appropriate acid such as, but not limited to mineral acid such as hydrochloric acid, sulfuric acid or hydrobromic acid, organic acids such as sulfonic acids or fluorinated sulfonic acids, an appropriate solvent with branched or unbranched carbon chain lengths of C1-C6 such as an alcohol, ketone, dimethyl formamide, diethyl formamide, dimethyl sulfoxide, dioxane, tetrahydrofuran with or without the addition of water. The reaction temperature may range from 0-120° C., more favorably 20-100° C.

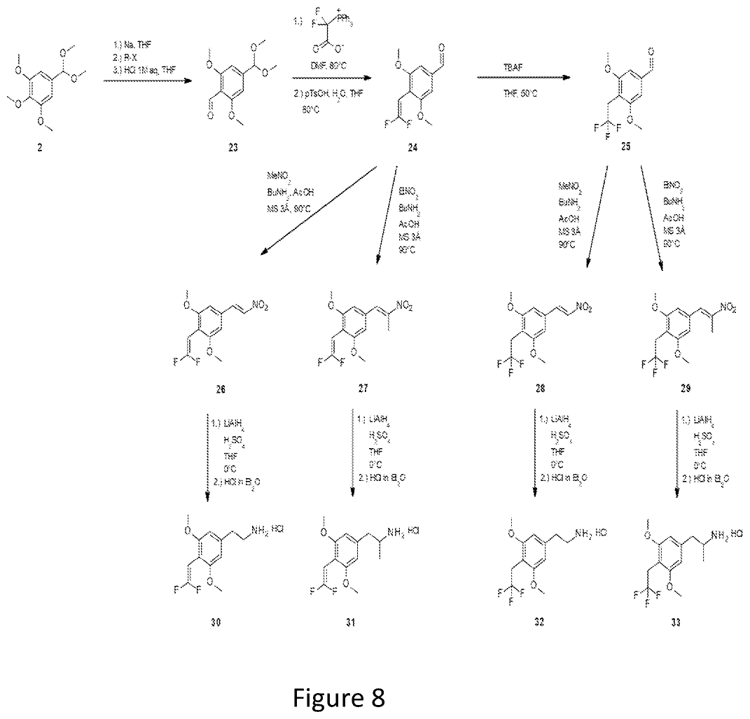

Aldehydes containing 4-fluorinated vinyl groups or 4-fluorinated alkylated substituents can also be accessed by the illustrative reaction of 3,5-dimethoxy-4-formyl-benzaldehyde dimethylacetal (23, FIG. 8) with (triphenylphosphonio)difluoroacetate in an organic solvent such as DMF or DMSO at elevated temperatures, favorably at 40-100° C., to form a difluorovinyl intermediate which can be carbonyl-deprotected to get the corresponding benzaldehyde 24. In a subsequent reaction with, e.g., tetrabutylammonium fluoride (TBAF) one can get the trifluoroethyl derivative 25, as illustrated in FIG. 8. The reaction sequence of carbonyl deprotection and reaction with TBAF can also be performed in a reversed order.

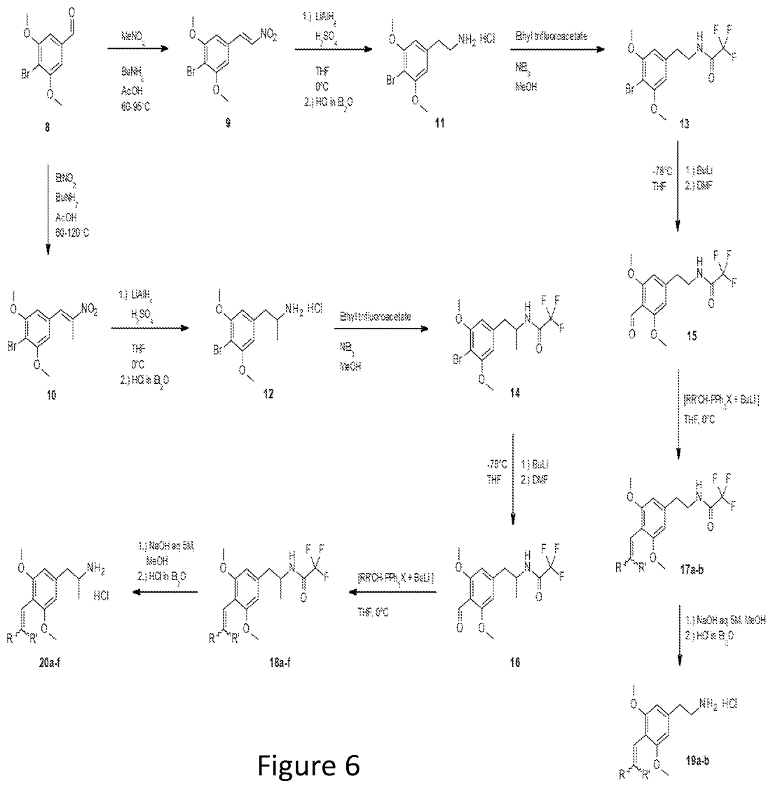

The 4-alkylated 3,5-dimethoxybenzaldehydes are then subjected to an aldol condensation, namely the Henry reaction, by mixing any of these aldehydes with a nitroalkane such as nitromethane, nitroethane or 1-nitropropane and a catalyst such as an organic salt or a mixture of an organic base and an organic acid, most favorably n-butylamine and acetic acid (such as illustrated in FIGS. 5, 6 and 8). The mixture can or not then be treated with heat in absence or presence of a drying agent such as an inorganic salt or, most favorably, molecular sieves. The water formed may also be removed azeotropically during reaction. The reaction mixture is cooled, and the product solids formed are filtered of, or the mixture is concentrated in vacuo prior further treatment. The obtained residue is further purified by crystallization or recrystallization or by column chromatography in order to get the final nitroolefins such as 4a-c and 5a-d (FIG. 5) as well as 9-10 (FIG. 6) and 26-29 (FIG. 8).

As continuation of the illustrative invention, the obtained nitroalkenes are dissolved in an inert solvent such as tetrahydrofuran or diethyl ether and added to a suspension of alane generated in situ from allowing to react lithium aluminum hydride (LiAlH₄) with concentrated sulfuric acid (H₂SO₄) in a similar solvent (such as illustrated in FIGS. 5, 6 and 8). The reaction temperature is set between −20° C. and 70° C., favorably at 0° C.-60° C. The reduction can also be performed by any other suitable conditions, such as the use of LiAlH₄ without H₂SO₄. The reaction mixture is then quenched subsequently with an alcohol, favorably isopropanol, and then with a base such as aqueous sodium hydroxide before filtering it off. The Filtrate is concentrated in vacuo and during the process an inert gas such as argon or nitrogen can be applied in order to prevent any carbamate formation. The residual desoxyscaline or 3C-desoxyscaline free base (such as of 6a-c and 7a-d as well as of 11-12 and 30-33, as illustrated in FIGS. 5, 6 and 8) is then dissolved in a solvent, favorably non-protic, most favorably in diethyl ether or dioxane, and neutralized by the addition of anhydrous hydrogen chloride or sulfuric acid or any other salt forming organic agent such as fumaric acid, tartaric acid, or acetic acid in a similar solvent.

In order to access alkene derivatives such as represented by compounds 19a-b and 20a-f (illustrated in FIG. 6), the compounds are prepared from the corresponding 3,5-dimethoxy-4-halophenethylamine or its amphetamine counterpart bearing a suitable protecting group on the basic nitrogen. Such protecting groups can be N-trifluoroacetyl or any other conditions-resistant group. In such a way, compounds 13 and 14 can be converted to the 4-formyl analogs by suitable conditions. As illustrated in FIG. 6, these conditions can consist of a metalation step by the use of a suitable agent such as butyllithium in a suitable organic inert solvent such as THF, dioxane or diethyl ether, at a reaction temperature of −150° C. to −0° C., more favorably at −120° C. to −70° C. Next, the metalated intermediates are treated with a formyl-introducing agent such as dimethylformamide. As illustrated in FIG. 6, the obtained 4-formyl derivatives 15 and 16 can then be used for C—C coupling reactions to form 4-alkene-containing compounds. As illustrative examples, a Wittig-type reaction can be applied, e.g., by the use of a Wittig salt, which is first converted to a suitable ylide by the use of, as illustrated in FIG. 6, a base such as butyllithium in a suitable organic inert solvent such as THF, dioxane or diethyl ether at a reaction temperature allowing the reaction to be sufficiently selective. More specifically, a temperature of −80° C. to 20° C. and more favorably at −20° C. to 10° C. is applied. The 4-formyl derivatives, such as 15 and 16, are then added to the ylides prepared as described before. Depending on the reaction conditions and reagents and additives used, one can force the reaction to yield a preferred amount of either of the configurational alkene isomers E or Z or cis or trans. Alternatively, the configurational isomers can also be separated by any suitable technique such as chromatography, crystallization, or distillation. To access the final compounds the protecting group is removed by known procedures. Illustrative, for N-trifluoroacetyl protected compounds (17a-b and 18a-f, FIG. 6), a suitable base can be aqueous sodium hydroxide in methanol, but any other suitable conditions can be applied. The residual desoxyscaline or 3C-desoxyscaline free base (such as of 19a-b and 20a-f, as illustrated in FIG. 6) is then converted to a suitable salt by conditions as described above.

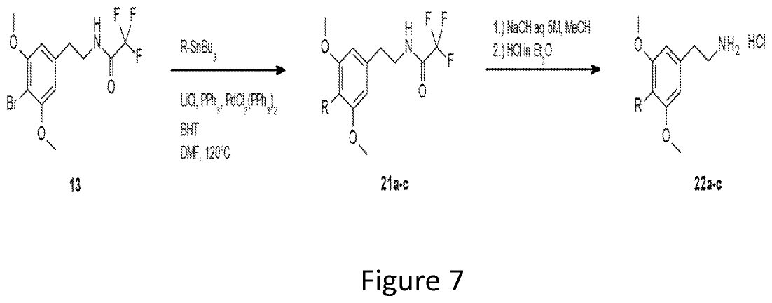

To access compounds represented in FIG. 1, an additional synthetic pathway can be followed, as illustrated in FIG. 7. N-Trifluoroacetyl protected 4-bromo-3,5-dimethoxyphenethylamine 13 (preparation see FIG. 6) can serve as another valuable building block. As such, it can be used for accessing the corresponding 4-aryl alkylated, alkenylated or alkynylated compounds by applying C—C Stille-type or any other C—C coupling reactions. As illustrative examples and outlined in FIG. 7, compound 13 was allowed to react with a set of organotin compounds (stannanes) under suitable conditions. Such conditions can apply the use of additives such as lithium chloride, triphenylphosphine, a transition metal catalyst such as bis(triphenylphosphine)palladium dichloride and a stabilizing agent such as 2,6-di-tert-butyl-4-methylphenol (BHT). The reaction is performed at reaction temperatures of 0-150° C., more favorably at 80-140° C., in a suitable solvent such as DMF or DMSO. The N-trifluoroacetyl protected products accessed by the routes described before, such as compounds 21a-c, could then be N-deprotected under conditions described above, in order to access final compounds such as 22a-c, as represented in FIG. 7. Conversion of the obtained free amine bases of, e.g., 22a-c to suitable salt forms can be performed as described above.

Detailed Description of the Chemical Preparation of the Compounds

General. NMR was performed on a Bruker NMR (¹H: 300 MHz and ¹⁹F: 282 MHz) at ambient temperature. Reaction controls were performed by silica gel TLC (F₂₅₄; UV detection) and HPLC UV & MS (Agilent 1100, Waters SQD).

General method for the ipso-substitution (4-demethoxy-alkylsubstitution). A solution of 4.00 g (17 mmol) 3,4,5-trimethoxybenzaldehyde dimethyl acetal (2) in 20 mL THF anhydr. Was added within 3 minutes to 1.2 g (52 mmol) freshly cut sodium in 60 mL THF anhydr. Under ice-cooling and a nitrogen atmosphere. The ice bath was removed, and the mixture was stirred for 22 hours whereby the color changed from clear to yellow and progressively to dark red. Next, the mixture was cooled with an ice-bath and 25 mmol neat alkyl halide were added over a course of 5 minutes whereby the color changed towards orange and a fine suspension was formed (NaX). The ice-bath was removed and stirring at ambient temperature was continued for 2 hours. In order to remove most of the remaining sodium solids, the mixture was carefully and quickly decanted to another flask applying a continuous nitrogen stream and further stirred under nitrogen and ice-cooling. Decomposition of excess sodium: the above remaining sodium metal was held under THF anhydr. (40 mL were added) and nitrogen and then were carefully decomposed by dropwise addition of 2.5 mL water in 2.5 mL THF under ice-cooling. After 30 minutes, no more sodium was visible, and the mixture was discarded. In the meantime, the above reaction mixture was quenched by adding dropwise 20 mL water. Next, 50 mL diethyl ether (Et₂O) were added and the layers were separated. The org. layer was washed once with water (20 mL) and brine (20 mL), dried over Na₂SO₄ and concentrated in vacuo to get the 4-alkyl-3,5-dimethoxybenzaldehyde dimethyl acetal as crude product. This was dissolved in 50 mL of THF/HCl 1M aq. 1:1 and stirred for 4 hours. The mixture was extracted with diethyl ether (3×30 mL) and the combined org. extracts were washed with water (2×30 mL), dried over Na₂SO₄ and concentrated in vacuo. The residue was either recrystallized or purified by silica gel chromatography.

General method for the nitro olefination (modified Henry reaction). The aryl aldehyde is dissolved in nitromethane or nitroethane under slight warming. Next, molecular sieves 3 Å (where applied), n-butylamine and acetic acid is added, and the mixture is gently stirred at 60-110° C. under an inert atmosphere. When the reaction is complete (monitoring by TLC, i.e., dichloromethane) the mixture is separated from the molecular sieves and concentrated in vacuo. The residue is either recrystallized from an appropriate solvent or purified by dissolving it in a small amount of organic solvent and eluting it with organic solvent through a short-path silica gel column. The eluate obtained is concentrated in vacuo. Usually, the trans-nitroolefin is obtained. In some cases, minor amounts of the cis-nitroolefin were observed as well; upon reduction (see next step) both cis and trans isomers lead to the same products and thus no separation is required.

General method for the alane-promoted reduction of the nitroolefins. To an ice-cooled suspension of lithium aluminum hydride (LiAlH₄) in tetrahydrofuran (THF) anhydr. Is added dropwise sulfuric acid (H₂SO₄) 95-99% under an inert atmosphere and vigorous stirring. When hydrogen evolution has ceased the mixture is stirred for another 5-10 minutes. Next, a solution of the nitroolefin in THF anh. is added under ice-cooling at such a rate that the reaction becomes not too violent, and the reaction temperature stays below 20-30° C. After completion of addition the mixture is brought to a gentle reflux for 3-5 minutes, and then again cooled with an ice-bath. Next, the mixture is cautiously quenched by successive and dropwise addition of anhydr. Isopropanol (IPA) and then 2M sodium hydroxide solution (NaOH). Occasionally, THF is added to keep the mixture stirable. When hydrolysis is complete, the mixture is filtered off and the filter cake is rinsed well with THF. The filtrate is concentrated in vacuo; purging the apparatus can be performed by applying an inert gas such as nitrogen or argon which prevents the formation of any unwanted carbamates.

General method for the hydrochloride salt formations. The base of the desoxyscaline or 3C-desoxyscaline is dissolved in approx. 30-50 times the mass of anhydr. Diethyl ether containing 0.5% anhydr. IPA. The well stirred solution is cautiously neutralized by the addition of 2M anhydr. HCl in diethyl ether or 4M anhydr. HCl in dioxane and occasional cooling; the pH should not be far from neutral in order to not get a sticky mass during processing. The suspension obtained is filtered off, rinsed with diethyl ether, and dried in vacuo to get the final hydrochloride product.

### Examples—Preparation of the Aldehydes 3a-d, 23, 24 and 25

3,4,5-Trimethoxybenzaldehyde dimethyl acetal, 2. To a stirred suspension of 0.60 g ammonium chloride in 80 mL methanol anhydr. And 80 mL trimethyl orthoformate were added 40.0 g (0.204 mol) 3,4,5-trimethoxybenzaldehyde under nitrogen in one portion. The mixture was heated to reflux for 2.5 hours. After cooling to approx. 10° C., 6.0 mL NEt₃ was added within 2 minutes, the mixture was further stirred for 5 minutes and then quenched with 200 mL water. The mixture was extracted with Et₂O (3×120 mL), and the combined org. extracts were washed with sat. aq. NaHCO₃ (1×120 mL) and water (1×120 mL) where the latter step should be performed quickly since there seemed to be a slight temperature increase during washing. The org. layer was dried over Na₂SO₄ and concentrated in vacuo at up to 60° C. to get 49.54 g (100.2%) crude product as an orangish oil. For further purification this could be distilled at 0.5 mbar (bath 230° C., head temp: 140-145° C.) but for several batches it was proven not to be necessary for the subsequent reactions. ¹H-NMR (DMSO-d6; Note: in CDCl₃ the product decomposes extremely quickly due to traces of HCl): 3.25 (s, 6H, CH(CH₃)₂), 3.66 (s, 1 MeO), 3.78 (s, 2MeO), 5.30 (s, CH(Ome)₂), 6.67 (s, 2 arom. H).

3,5-Dimethoxy-4-methylbenzaldehyde, 3a. Adapted from (Comins et al., 1984). To a solution of 0.670 mL (6.6 mmol, 1.1 eq) N-methylpiperazine in 20 mL THF anhydr. Were added 2.55 mL (6.3 mmol, 1.05 eq) butyl lithium (BuLi) 2.5M within 2 minutes under nitrogen at −20° C. After stirring for 15 minutes a solution of 1.00 g (6.01 mmol) 3,5-dimethoxybenzaldehyde in 5 mL THF anhydr. Was added within 2 minutes and stirring at this temperature was continued for 20 minutes. Next, 7.2 mL (18.1 mmol, 3 eq) BuLi 2.5M were added within 4 minutes and the clear colorless solution was allowed to stand at −20° C. for 24 hours (freezer). The slight yellowish clear solution was re-submerged into a cooling bath at −20° C., and under stirring 2.25 mL (36.1 mmol, 6 eq) neat methyl iodide was added dropwise (3 minutes) whereby the mixture remained clear. After 30 minutes the cooling bath was removed, and the mixture allowed to reach ambient temperature; during this time, the mixture became gel-like and occasionally was non-stirrable and a sticky mass was formed on the bottom. Next, 50 mL ice-cold HCl 2M and then 30 mL Et₂O were added, and the layers were separated. The organic layer was washed subsequently with water (3×20 mL) and brine (1×20 mL), dried over MgSO₄ and concentrated in vacuo to get 1.07 g (99%) of a yellowish solid with sufficient purity. ¹H-NMR (CDCl₃): 2.18 (s, ArCH₃), 3.92 (s, 2MeO), 7.08 (s, 2 arom. H), 9.93 (s, CHO).

3,5-Dimethoxy-4-ethylbenzaldehyde, 3b. According to the general method described (ipso-substitution), 3.19 g crude aldehyde were obtained. This was further purified by silica gel chromatography (coated on silica gel using a minimum amount of dichloromethane, elution with hexane/ethyl acetate 9/1). Yield: 1.67 g (50.6%) product 3b as a white solid. ¹H-NMR (CDCl₃): 1.11 (t, CH₂CH₃), 2.73 (q, CH₂CH₃), 3.91 (s, 2MeO), 7.08 (s, 2 arom. H), 9.92 (s, CHO).

3,5-Dimethoxy-4-propylbenzaldehyde, 3c. According to the general method described (ipso-substitution; 1.8 times higher scale), 6.1 g crude aldehyde were obtained. This was further purified by silica gel chromatography (coated on silica gel using a minimum amount of dichloromethane, elution with hexane/ethyl acetate 9/1). Yield: 3.42 g (54.2%) product 3c as a white solid. ¹H-NMR (CDCl₃): 0.93 (t, CH₂CH₂CH₃), 1.52 (m, CH₂CH₂CH₃), 2.67 (t, CH₂CH₂CH₃), 3.86 (s, 2MeO), 7.05 (s, 2 arom. H), 9.90 (s, CHO).

4-Allyl-3,5-dimethoxybenzaldehyde, 3d. According to the general method described (ipso-substitution; 1.25 times higher scale), 4.56 g crude aldehyde were obtained. This was further purified by silica gel chromatography (coated on silica gel using a minimum amount of dichloromethane, elution with hexane/ethyl acetate 995/5). Yield: 0.40 g (9.4%) product 3d as a yellowish oil. ¹H-NMR (CDCl₃): 3.48 (dm, ArCH₂), 3.92 (s, 2MeO), 4.95-5.04 (m, 2H, H₂C═C), 5.93 (m, H₂C═CH), 7.10 (s, 2 arom. H), 9.94 (s, CHO).

4-(2,2-Difluorovinyl)-3,5-dimethoxybenzaldehyde, 24. 1.) Preparation of the intermediate 23. A solution of 7.35 g (30.33 mmol) 3,4,5-trimethoxybenzaldehyde dimethyl acetal (2) in 30 mL THF anhydr. Was added within 3 minutes to 2.13 g (92.8 mmol) freshly cut sodium in 130 mL THF anhydr. Under ice-cooling and a nitrogen atmosphere. The ice bath was removed, and the mixture was stirred for 20 hours whereby the color changed from clear to yellow and progressively to dark red. Next, the mixture was cooled with an ice-bath and 3.52 mL neat DMF anhydr. (45.5 mmol) were added over a course of 5 minutes. The ice-bath was removed and stirring at ambient temperature was continued for 1 hour. In order to remove most of the remaining sodium solids, the mixture was carefully and quickly decanted to another flask and further stirred under nitrogen and ice-cooling. Decomposition of excess sodium: the above remaining sodium metal was held under THF anhydr. (60 mL were added) and nitrogen and then were carefully decomposed by dropwise addition of 5 mL water in 5 mL THF under ice-cooling. After some 30 minutes no more sodium was visible, and the mixture was discarded. In the meantime, the above reaction mixture was quenched by adding dropwise 70 mL water. Next, 100 mL tert-butylmethylether (TBME) were added and the layers were separated; to ease phase separation some 50 mL sat. NaHCO₃ solution were added. The aq. Layer was extracted with additional TBME (3×50 mL), and the combined org. layers were dried over Na₂SO₄ and concentrated in vacuo to get 6.33 g yellow solid. This was triturated in 10 mL ice-cold isopropanol, filtered off and the filter cake was rinsed with a minimal amount of additional ice-cold isopropanol and dried in vacuo to get 3.66 g (50.3%) of the intermediate 3,5-dimethoxy-4-formylbenzaldehyde dimethyl acetal (23) as a white solid. ¹H-NMR (DMSO-d6; Note: in CDCl₃ the product decomposes extremely quickly due to traces of HCl): 3.29 (s, 6H, CH(CH₃)₂), 3.84 (s, 2MeO), 5.37 (s, CH(Ome)₂), 6.73 (s, 2 arom. H), 10.34 (s, CHO). 2.) Preparation of the Wittig reagent (triphenylphosphonio)difluoroacetate (Zheng et al., 2013): In a 250 mL flask, 31.5 g (120 mmol) triphenylphosphine and 25.6 g (120 mmol) potassium bromodifluoroacetate were placed and mixed with DMF anhydr. Under nitrogen at ambient temperature. The solution was stirred for 23 hours whereby a suspension was formed progressively (product plus KBr). The suspension was filtered off, and the solid was washed with DMF (2×10 mL), H₂O (2×10 mL) and Et₂O (3×20 mL), and finally dried in vacuo at 40° C. for 20 hours to get 34.1 g (79.8%) (triphenylphosphonio)difluoroacetate as a white powder. ¹H-NMR (CD₃OD) 7.75-7.81 (m, 6H), 7.86-7.98 (m, 9H). ¹⁹F-NMR (CD₃OD) −96.00 (d, 2F, J=95.9 Hz). 3.) Wittig reaction to access aldehyde 24. A mixture of 1.8 g (7.49 mmol) 3,5-dimethoxy-4-formylbenzaldehyde dimethyl acetal (23) and 5.4 g (15.0 mmol) (triphenylphosphonio)difluoroacetate in 20 mL DMF anhydr. Was heated to 80° C. under nitrogen for 16 hours. Next, the mixture was cooled to ambient temperature and diluted with 150 mL dichloromethane (DCM), washed with water (2×70 mL), half-saturated NaHCO₃ solution (1×70 mL) and again with water (1×70 mL). The org. layer was dried over Na₂SO₄, directly filtered through a small silica gel pad (ca. 1 cm height) and the pad was rinsed with additional DCM. The combined filtrates were concentrated in vacuo to get 6.4 g crude 4-(2,2-difluorovinyl)-3,5-dimethoxybenzaldehyde dimethyl acetal as intermediate to be used directly in the next step. Thus, the residue was dissolved in 50 mL THF, and then 100 mL water and 50 mg pTsOH were added. The mixture was vigorously stirred at 80° C. for 30 min, cooled to ambient temperature and diluted with 150 mL ethyl acetate (EtOAc). The layers were separated, and the org. layer was washed with water (4×50 mL), dried over Na₂SO₄ and concentrated in vacuo. The residue was purified by silica gel chromatography (hexane/EtOAc, from 95/5 to 7/1) to get 1.68 g (98.2%) product 24 as a white solid. ¹H-NMR (CDCl₃): 3.93 (s, 2MeO), 5.25 (dd, ³J(H,F)=27 Hz and 3.0 Hz, F₂CCH), 7.09 (s, 2 arom. H), 9.93 (s, CHO). ¹⁹F-NMR (CDCl₃): −74.83 (d, 1F, J=19.7 Hz), −82.27 (d, 1F, J=19.7 Hz).

3,5-Dimethoxy-4-(2,2,2-trifluoroethyl)benzaldehyde, 25. To a solution of 1.54 g (6.75 mmol) 4-(2,2-difluorovinyl)-3,5-dimethoxybenzaldehyde in 7 mL THF anhydr. Were added 7.0 mL 1M tetrabutylammonium fluoride (TBAF) in THF and the mixture was heated to 50° C. under nitrogen. After 1.5 hours another 0.7 mL 1M TBAF in THF were added and heating was continued for another 2.5 hours. Next, the mixture was cooled to ambient temperature, poured into 50 mL water and extracted with EtOAc (1×100 mL). The org. layer was washed with water (2×50 mL), dried over Na₂SO₄ and concentrated in vacuo. The residue was dissolved in a minimal amount of DCM and purified by silica gel chromatography (hexane/EtOAc, from 9/1 to 4/1) to get 0.910 g (51.9%) product 25 as a white solid. ¹H-NMR (CDCl₃): 3.58 (q, ³J(H,F)=10.8 Hz, F₃CCH₂), 3.92 (s, 2MeO), 7.09 (s, 2 arom. H), 9.94 (s, CHO). ¹⁹F-NMR (CDCl₃): −64.51 (s, 3F).

### Examples—Preparation of the Nitroolefins 4a-c, 5a-d, 9-10 and 26-29

3,5-Dimethoxy-4-methyl-β-nitrostyrene, 4a. According to the general method described, from 0.55 g 3a, 0.6 mL nitromethane, 22 μL butylamine, 22 μL acetic acid and 0.30 g molecular sieves, 30 minutes at 90° C. Yield: 0.33 g (48.5%) 4a as a yellow solid. ¹H-NMR (CDCl₃): 2.15 (s, ArCH₃), 3.89 (s, 2MeO), 6.71 (s, 2 arom. H), 7.60 (d, CHNO₂), 7.99 (d, CH═CHNO₂). A small second signal set (˜4%) was observed and corresponded to the correlative cis-nitroolefin (this phenomenon has been observed and proven earlier by one of the authors (Trachsel, 2002)). Upon reduction, this leads to the same product as the trans-isomer and thus it was not necessary to remove.

1-(3,5-Dimethoxy-4-methylphenyl)-2-nitropropene, 5a. According to the general method described, from 0.48 g 3a, 0.50 mL nitroethane, 204 butylamine, 204 acetic acid and 0.20 g molecular sieves, 90 minutes at 90° C. Yield: 0.34 g (53.9%) 5a as a yellow solid. ¹H-NMR (CDCl₃): 2.12 (s, ArCH₃), 2.49 (d, MeC), 3.85 (s, 2MeO), 6.59 (s, 2 arom. H), 8.07 (s, CH═C). A small second signal set (˜5%) was observed and corresponded to the correlative cis-nitroolefin (this phenomenon has been observed and proven earlier by one of the authors (Trachsel, 2002)). Upon reduction, this leads to the same product as the trans-isomer and thus it was not necessary to remove.

3,5-Dimethoxy-4-ethyl-β-nitrostyrene, 4b. According to the general method described, from 0.92 g 3b, 1.0 mL nitromethane, 364 butylamine, 364 acetic acid and 0.40 g molecular sieves, 40 minutes at 90° C. Yield: 0.96 g (85.5%) 4b as a yellow solid. ¹H-NMR (CDCl₃): 1.10 (t, CH₂CH₃), 2.71 (q, CH₂CH₃), 3.88 (s, 2MeO), 6.71 (s, 2 arom. H), 7.60 (d, CHNO₂), 7.99 (d, CH═CHNO₂). A small second signal set (˜5%) was observed and corresponded to the correlative cis-nitroolefin (this phenomenon has been observed and proven earlier by one of the authors (Trachsel, 2002)). Upon reduction, this will lead to the same product as the trans-isomer and thus it was not necessary to remove.

1-(3,5-Dimethoxy-4-ethylphenyl)-2-nitropropene, 5b. According to the general method described, from 0.70 g 3b, 0.70 mL nitroethane, 284 butylamine, 284 acetic acid and 0.35 g molecular sieves, 90 minutes at 90° C. Yield: 0.67 g (74%) 5b as a yellow solid. ¹H-NMR (CDCl₃): 1.09 (t, CH₂CH₃), 2.49 (d, MeC), 2.68 (q, CH₂CH₃), 3.84 (s, 2MeO), 6.60 (s, 2 arom. H), 8.07 (s, CH═C). A small second signal set (˜6%) was observed and corresponded to the correlative cis-nitroolefin (this phenomenon has been observed and proven earlier by one of the authors (Trachsel, 2002). Reduction, this leads to the same product as the trans-isomer and thus it was not necessary to remove.

3,5-Dimethoxy-4-propyl-β-nitrostyrene, 4c. According to the general method described, from 2.00 g 3c, 2.0 mL nitromethane, 62 μL butylamine, 62 μL acetic acid and 1.0 g molecular sieves, 40 minutes at 90° C. Yield: 2.04 g (84.6%) 4c as a yellow solid. 1H-NMR (CDCl₃): 0.95 (t, CH₂CH₂CH₃), 1.51 (m, CH₂CH₂CH₃), 2.64 (t, CH₂CH₂CH₃), 3.87 (s, 2MeO), 6.68 (s, 2 arom. H), 7.58 (d, CHNO₂), 7.97 (d, CH═CHNO₂). A small second signal set (˜8%) was observed and corresponded to the correlative cis-nitroolefin (this phenomenon has been observed and proven earlier by one of the authors (Trachsel, 2002)). Upon reduction, this leads to the same product as the trans-isomer and thus it was not necessary to remove.

1-(3,5-Dimethoxy-4-propylphenyl)-2-nitropropene, 5c. According to the general method described, from 1.30 g 3c, 1.3 mL nitroethane, 52 μL butylamine, 52 μL acetic acid and 0.7 g molecular sieves, 105 minutes at 90° C. Yield: 1.09 g (65.8%) 5c as a yellow solid. ¹H-NMR (CDCl₃): 0.99 (t, CH₂CH₂CH₃), 1.53 (m, CH₂CH₂CH₃), 2.52 (d, MeC), 2.66 (t, CH₂CH₂CH₃), 3.86 (s, 2MeO), 6.62 (s, 2 arom. H), 8.08 (s, CH═C). A small second signal set (˜7%) was observed and corresponded to the correlative cis-nitroolefin (this phenomenon has been observed and proven earlier by one of the authors (Trachsel, 2002)). Upon reduction, this leads to the same product as the trans-isomer and thus it was not necessary to remove.

1-(4-Allyl-3,5-dimethoxyphenyl)-2-nitropropene, 5d. According to the general method described, from 0.38 g 3d, 0.40 mL nitroethane, 15 μL butylamine, 15 μL acetic acid and 0.20 g molecular sieves, 90 minutes at 90° C. Yield: 0.30 g (62.0%) 5d as a yellow solid. ¹H-NMR (CDCl₃): 2.49 (d, MeC), 3.43 (dm, ArCH₂), 3.84 (s, 2MeO), 4.92-5.03 (m, 2H, H₂C═C), 5.92 (m, H₂C═CH), 6.60 (s, 2 arom. H), 8.06 (s, CH═C). A small second signal set (˜3%) was observed and corresponded to the correlative cis-nitroolefin (this phenomenon has been observed and proven earlier by one of the authors (Trachsel, 2002)). Upon reduction, this leads to the same product as the trans-isomer and thus it was not necessary to remove.

4-Bromo-3,5-dimethoxy-β-nitrostyrene, 9. According to the general method described, from 15.0 g 4-bromo-3,5-dimethoxybenzaldehyde (8; commercially available), 25 mL nitromethane, 0.60 mL butylamine, 0.60 mL acetic acid and 2.5 g molecular sieves, 35 minutes at 90° C. Yield: 16.1 g (89.8%) 9 as a pale-yellow solid. ¹H-NMR (CDCl₃): 3.98 (s, 2MeO), 6.73 (s, 2 arom. H), 7.62 (d, CHNO₂), 7.97 (d, CH═CHNO₂). A second signal set (˜20%) was observed and corresponded to the correlative cis-nitroolefin (this phenomenon has been observed and proven earlier by one of the authors (Trachsel, 2002)): 3.94 (s, 2MeO), 6.85 (s, 2 arom. H), 7.62 (d, CHNO₂), 8.39 (d, CH═CHNO₂). Upon reduction, this leads to the same product as the trans-isomer and thus it was not necessary to remove.

1-(4-Bromo-3,5-dimethoxyphenyl)-2-nitropropene, 10. According to the general method described, from 20.0 g 4-bromo-3,5-dimethoxybenzaldehyde (8; commercially available), 33 mL nitroethane, 0.80 mL butylamine, 0.80 mL acetic acid and 2.5 g molecular sieves, 90 minutes at 90° C. Yield: 20.29 g (81%) 10 as a pale-yellow solid. ¹H-NMR (CDCl₃): 2.46 (d, MeC), 3.92 (s, 2MeO), 6.59 (s, 2 arom. H), 8.02 (s, CH═C). No cis-nitroolefin was apparent.

4-(2,2-Difluorovinyl)-3,5-dimethoxy-β-nitrostyrene, 26. According to the general method described, from 0.75 g 24, 0.80 mL nitromethane, 24 μL butylamine, 24 μL acetic acid and 0.40 g molecular sieves, 60 minutes at 90° C. Yield: 0.58 g (65.1%) 26 as a yellow solid. ¹H-NMR (CDCl₃): 3.91 (s, 2MeO), 5.23 (dd, ³J(H,F)=27.3 Hz and 2.7 Hz, F₂CCH), 6.72 (s, 2 arom. H), 7.61 (d, CHNO₂), 7.98 (d, CH═CHNO₂). ¹⁹F-NMR (CDCl₃): −75.18 (d, 1F, J=19.7 Hz), −82.50 (d, 1F, J=19.7 Hz). No cis-nitroolefin was apparent.

1-(4-(2,2-Difluorovinyl)-3,5-dimethoxyphenyl)-2-nitropropene, 27. According to the general method described, from 0.75 g 24, 0.80 mL nitroethane, 24 μL butylamine, 24 μL acetic acid and 0.4 g molecular sieves, 80 minutes at 90° C. Yield: 0.45 g (48%) 27 as a yellow solid. ¹H-NMR (CDCl₃): 2.51 (d, MeC), 3.89 (s, 2MeO), 5.23 (dd, ³J(H,F)=27.3 Hz and 2.7 Hz, F₂CCH), 6.62 (s, 2 arom. H), 8.07 (s, CH═C). ¹⁹F-NMR (CDCl₃): −76.19 (d, 1F, J=22.6 Hz), −83.20 (d, 1F, J=22.6 Hz). No cis-nitroolefin was apparent.

3,5-Dimethoxy-4-(2,2,2-trifluoroethyl)-β-nitrostyrene, 28. According to the general method described, from 0.45 g 25, 0.50 mL nitromethane, 13 μL butylamine, 13 μL acetic acid and 0.20 g molecular sieves, 60 minutes at 90° C. Yield: 0.36 g (68.2%) 28 as a yellow solid. ¹H-NMR (CDCl₃): 3.58 (q, ³J(H,F)=10.5 Hz, F₃CCH₂), 3.91 (s, 2MeO), 6.74 (s, 2 arom. H), 7.60 (d, CHNO₂), 7.98 (d, CH═CHNO₂). ¹⁹F-NMR (CDCl₃): −64.63 (s, 3F). No cis-nitroolefin was apparent.

1-(3,5-Dimethoxy-4-(2,2,2-trifluoroethyl)phenyl)-2-nitropropene, 29. According to the general method described, from 0.45 g 25, 0.50 mL nitroethane, 13 μL butylamine, 13 μL acetic acid and 0.20 g molecular sieves, 2.5 hours at 90° C. Yield: 0.40 g (72.2%) 29 as a yellow solid. ¹H-NMR (CDCl₃): 2.50 (d, MeC), 3.57 (q, ³J(H,F)=10.5 Hz, F₃CCH₂), 3.89 (s, 2MeO), 6.62 (s, 2 arom. H), 8.07 (s, CH═C). ¹⁹F-NMR (CDCl₃): −64.74 (s, 3F). No cis-nitroolefin was apparent.

### Examples—Alane-Promoted Reduction of the Nitroolefins to the Amines and Conversion to their Salts: Preparation of the Desoxyscalines and 3C-Desoxyscalines 6a-c, 7a-d, 11-12 and 30-33

3,5-Dimethoxy-4-methylphenethylamine hydrochloride (D; Desoxyscaline), 6a. According to the general method described, from 0.32 g 4a, 0.20 g LiAlH₄, 0.14 mL H₂SO₄, 4 mL plus 2 mL THF, 0.85 mL IPA and 0.65 mL NaOH 2M. Hydrochloride salt formation according to the general method described. Yield: 0.16 g (48.3%) product as a white solid. ¹H-NMR (D₂O): 1.97 (s, ArCH₃), 2.91 (t, ArCH₂), 3.22 (t, CH₂NH₃⁺), 3.79 (s, 2 MeO), 6.61 (s, 2 arom. H).

3,5-Dimethoxy-4-methylamphetamine hydrochloride (3C-D), 7a. According to the general method described, from 0.32 g 5a, 0.19 g LiAlH₄, 0.13 mL H₂SO₄, 4 mL plus 2 mL THF, 0.90 mL IPA and 0.70 mL NaOH 2M. Hydrochloride salt formation according to the general method described. Yield: 0.19 g (57.3%) product as a white solid. ¹H-NMR (D₂O): 1.34 (d, MeCH), 2.07 (s, ArCH₃), 2.94 (d, ArCH₂), 3.68 (m, CHNH₃+), 3.88 (s, 2 MeO), 6.67 (s, 2 arom. H).

3,5-Dimethoxy-4-ethylphenethylamine hydrochloride (DE; Desoxyescaline), 6b. According to the general method described, from 0.94 g 4b, 0.56 g LiAlH₄, 0.39 mL H₂SO₄, 13 mL plus 5 mL THF, 2.3 mL IPA and 1.8 mL NaOH 2M. Hydrochloride salt formation according to the general method described. Yield: 0.47 g (48.3%) product as a white solid. ¹H-NMR (D₂O): 1.04 (t, ArCH₂CH₃), 2.61 (q, ArCH₂CH₃), 2.99 (t, ArCH₂), 3.30 (t, CH₂NH₃+), 3.87 (s, 2MeO), 6.70 (s, 2 arom. H).

3,5-Dimethoxy-4-ethylamphetamine hydrochloride (3C-DE), 7b. According to the general method described, from 0.67 g 5b, 0.38 g LiAlH₄, 0.27 mL H₂SO₄, 10 mL plus 5 mL THF, 1.57 mL IPA and 1.20 mL NaOH 2M. Hydrochloride salt formation according to the general method described. Yield: 0.50 g (74.1%) product as a white solid. ¹H-NMR (D₂O): 1.04 (t, ArCH₂CH₃), 1.33 (d, MeCH), 2.61 (q, ArCH₂CH₃), 2.93 (d, ArCH₂CH), 3.67 (m, CHNH₃+), 3.86 (s, 2MeO), 6.66 (s, 2 arom. H).

3,5-Dimethoxy-4-propylphenethylamine hydrochloride (DP; Desoxyproscaline), 6c. According to the general method described, from 2.0 g 4c, 1.13 g LiAlH₄, 0.79 mL H₂SO₄, 25 mL plus 6 mL THF, 4.7 mL IPA and 3.6 mL NaOH 2M. Hydrochloride salt formation according to the general method described. Yield: 1.57 g (76.9%) product as a white solid. ¹H-NMR (D₂O): 0.91 (t, CH₂CH₂CH₃), 1.48 (m, CH₂CH₂CH₃), 2.56 (t, CH₂CH₂CH₃), 2.99 (t, ArCH₂), 3.31 (t, CH₂NH₃+), 3.85 (s, 2MeO), 6.70 (s, 2 arom. H).

3,5-Dimethoxy-4-propylamphetamine hydrochloride (3C-DP), 7c. According to the general method described, from 1.05 g 5c, 0.56 g LiAlH₄, 0.39 mL H₂SO₄, 12 mL plus 5 mL THF, 2.3 mL IPA and 1.8 mL NaOH 2M. Hydrochloride salt formation according to the general method described. Yield: 0.84 g (77.7%) product as a white solid. ¹H-NMR (D₂O): 0.91 (t, CH₂CH₂CH₃), 1.33 (d, MeCH), 1.47 (m, CH₂CH₂CH₃), 2.56 (t, CH₂CH₂CH₃), 2.93 (d, ArCH₂CH), 3.66 (m, CHNH₃+), 3.85 (s, 2MeO), 6.67 (s, 2 arom. H).

4-Allyl-3,5-dimethoxyamphetamine hydrochloride (3C-DAL), 7d. According to the general method described, from 0.29 g 5d, 0.16 g LiAlH₄, 0.11 mL H₂SO₄, 4 mL plus 2 mL THF, 0.8 mL IPA and 0.6 mL NaOH 2M. Hydrochloride salt formation according to the general method described. Yield: 41 mg (13.7%) product as a white solid. ¹H-NMR (D₂O): 1.25 (d, MeCH), 2.95 (d, ArCH₂CHNH₃+), 3.40 (dm, ArCH₂CH═CH₂), 3.69 (m, CHNH₃+), 3.86 (s, 2MeO), 4.86-5.03 (m, 2H, H₂C═C), 6.03 (m, H₂C═CH), 6.69 (s, 2 arom. H).

4-Bromo-3,5-dimethoxyphenethylamine hydrochloride (DBR; Desoxybromoscaline), 11. According to the general method described, from 16.0 g 9 (added portion wise as a solid), 7.85 g LiAlH₄, 5.49 mL H₂SO₄, 250 mL THF, 32.6 mL IPA and 25 mL NaOH 2M. Hydrochloride salt formation according to the general method described. Yield: 11.91 g (72.3%) product as a white solid. ¹H-NMR (D₂O): 2.92 (t, ArCH₂), 3.24 (t, CH₂NH₃+), 3.83 (s, 2MeO), 6.64 (s, 2 arom. H).

4-Bromo-3,5-dimethoxyamphetamine hydrochloride (3C-DBR), 12. According to the general method described, from 20.2 g 10, 9.52 g LiAlH₄, 6.6 mL H₂SO₄, 200 mL plus 100 mL THF, 39.5 mL IPA and 30.2 mL NaOH 2M. Hydrochloride salt formation according to the general method described. Yield: 16.1 g (77.5%) product as a white solid. ¹H-NMR (D₂O): 1.32 (d, MeCH), 2.93 (m, ArCH₂CH), 3.67 (m, CHNH₃+), 3.89 (s, 2MeO), 6.63 (s, 2 arom. H).

4-(2,2-Difluorovinyl)-3,5-dimethoxyphenethylamine hydrochloride (DDFV; Desoxydifluorovinylscaline), 30. According to the general method described, from 0.56 g 26, 0.29 g LiAlH₄, 0.20 mL H₂SO₄, 7 mL plus 3 mL THF, 0.50 mL IPA and 0.38 mL NaOH 2M. Hydrochloride salt formation according to the general method described. Yield: 0.237 g (41.0%) product as a white solid. ¹H-NMR (D₂O): 3.02 (t, ArCH₂), 3.32 (t, CH₂NH₃+), 3.88 (s, 2MeO), 5.26 (dd, ³J(H,F)=27.6 Hz and 2.4 Hz, F₂CCH), 6.72 (s, 2 arom. H). ¹⁹F-NMR (D₂O): −80.38 (d, 1F, J=28.2 Hz), −85.07 (d, 1F, J=28.2 Hz).

4-(2,2-Difluorovinyl)-3,5-dimethoxyamphetamine hydrochloride (3C-DDFV), 31. According to the general method described, from 0.42 g 27, 0.21 g LiAlH₄, 0.15 mL H₂SO₄, 6 mL plus 3 mL THF, 0.50 mL IPA and 0.4 mL NaOH 2M. Hydrochloride salt formation according to the general method described. Yield: 0.30 g (68.2%) product as a white solid. ¹H-NMR (D₂O): 1.34 (d, MeCH), 2.07 (s, ArCH₃), 2.94 (d, ArCH₂), 3.69 (m, CHNH₃+), 3.87 (s, 2MeO), 5.25 (dd, ³J(H,F)=27.9 Hz and 2.1 Hz, F₂CCH), 6.69 (s, 2 arom. H). ¹⁹F-NMR (D₂O): −80.31 (d, 1F, J=31.0 Hz), −85.03 (d, 1F, J=31.0 Hz).

3,5-Dimethoxy-4-(2,2,2-trifluoroethyl)phenethylamine hydrochloride (DTFE; Desoxytrifluoroescaline), 32. According to the general method described, from 0.35 g 28, 0.171 g LiAlH₄, 0.12 mL H₂SO₄, 4 mL plus 2 mL THF, 0.70 mL IPA and 0.55 mL NaOH 2M. Hydrochloride salt formation according to the general method described. Yield: 0.14 g (38.9%) product as a white solid. ¹H-NMR (D₂O): 3.03 (t, ArCH₂), 3.33 (t, CH₂NH₃+), 3.60 (q, CH₂CF₃), 3.89 (s, 2MeO), 6.74 (s, 2 arom. H). ¹⁹F-NMR (D₂O): −64.59 (s, 3F).

3,5-Dimethoxy-4-(2,2,2-trifluoroethyl)amphetamine hydrochloride (3C-DTFE), 33. According to the general method described, from 0.38 g 29, 0.177 g LiAlH₄, 0.12 mL H₂SO₄, 4 mL plus 2 mL THF, 0.40 mL IPA and 0.31 mL NaOH 2M. Hydrochloride salt formation according to the general method described. Yield: 0.29 g (75%) product as a white solid. ¹H-NMR (D₂O): 1.34 (d, MeCH), 2.97 (d, ArCH₂), 3.61 (q, CH₂CF₃), 3.66 (m, CHNH₃+), 3.88 (s, 2MeO), 6.71 (s, 2 arom. H). ¹⁹F-NMR (D₂O): −64.92 (s, 3F).

### Examples—Preparation of the Desoxyscalines 19a-b and 3C-Desoxyscalines 20a-f Via Wittig Reaction and Conversion to their Salts

### 1. N-TFA Introduction

N-Trifluoroacetyl-4-bromo-3,5-dimethoxyphenethylamine, 13. To a solution of 7.40 g (24.94 mmol) 11 and 7.64 mL (54.87 mmol; 2.2 eq) NEt₃ in 55 mL MeOH anhydr. Was added dropwise 2.96 mL (27.44 mmol; 1.1 eq) ethyl trifluoroacetate within 1 minutes under nitrogen. After stirring for 1 hour at ambient temperature, the volatiles were removed in vacuo and the residue was redissolved in MTBE (150 mL), washed with aq. HCl 0.5M (2×100 mL) and water (2×50 mL), dried over Na₂SO₄ and concentrated in vacuo to get 7.58 g (85.3%) product 13 as a beige solid. ¹H-NMR (CDCl₃) revelated an approx. 90:10 mixture of cis/trans amide mixture (isomers not assigned; major product peak's shifts given first): 2.90/3.07 (t, ArCH₂), 3.67 (q (superimposed txd), CH₂NH), 3.91/3.89 (s, 2MeO), 6.34 (bs, NH), 6.41/6.58 (s, 2 arom. H).

N-Trifluoroacetyl-4-bromo-3,5-dimethoxyamphetamine, 14. To a solution of 15.50 g (49.9 mmol) 12 and 15.29 mL (109.8 mmol; 2.2 eq) NEt₃ in 110 mL MeOH anhydr. Was added dropwise 5.92 mL (54.88 mmol; 1.1 eq) ethyl trifluoroacetate within 1 minute under nitrogen. After stirring 1 hour at ambient temperature, the volatiles were removed in vacuo and the residue was redissolved in MTBE (300 mL) and ethyl acetate (300 mL), washed with aq. HCl 0.5M (2×200 mL) and water (2×100 mL), dried over Na₂SO₄ and concentrated in vacuo to get 16.26 g (88.0%) product 14 as a beige solid. ¹H-NMR (CDCl₃): 1.25 (d, MeCH), 2.84 (m, ArCH₂), 3.89 (s, 2MeO), 4.30 (m, CHNH), 6.11 (bs, NH), 6.36 (s, 2 arom. H).

### 2. Introduction of the Carbonyl Group

N-Trifluoroacetyl-3,5-dimethoxy-4-formylphenethylamine, 15. To a solution of 17 mL (42.14 mmol, 3.0 eq) BuLi 2.5M in 40 mL THF anhydr. At −100° C. (cooling bath: acetone/THF approx. 4/1 mixture, adjusted with liquid N₂) under nitrogen was added dropwise (10 minutes) a solution of 5.0 g (14.14 mmol) 13 in 60 mL THF anhydr. In such a manner that a “precooling” on the flask's wall occurred (needle touched the flask's wall). After stirring for 4 minutes, 16.8 mL DMF anhydr. Were added within 5 minutes in a similar way. After completion of addition stirring was continued for 45 minutes and then the mixture was allowed to reach approx. −10° C. Next, the mixture was quenched by a quick addition of 100 mL aq. Saturated NH₄Cl solution and 100 mL of citric acid 10%. The mixture was extracted with EtOAc (2×100 mL), and the combined org. extracts were washed twice with water, dried over Na₂SO₄ and concentrated in vacuo to get 3.9 g of an orange oil that crystallized upon cooling to ambient temperature. The residue was dissolved in 15 mL EtOAc under heating, and the solution was plugged onto a silica gel dry flash column (h: 4 cm, d: 6 cm) preconditioned with hexane/EtOAc 3:1. It was eluted with the same eluent until all the unwanted byproduct (N-trifluoroacetyl-3,5-dimethoxyphenethylamine) was eluted, then it was changed to hexane/EtOAc 1:2 in order to elute the desired product. After pooling and evaporating the corresponding fractions there were obtained 2.21 g (51.5%) product 15 as a beige solid. ¹H-NMR (CDCl₃) revelated an approx. 90:10 mixture of cis/trans amide mixture (isomers not assigned; major product peak's shifts given first): 2.93/3.11 (t, ArCH₂), 3.69 (q (superimposed txd), CH₂NH), 3.91/3.92 (s, 2MeO), 6.40/6.64 (s, 2 arom. H), 6.69 (bs, NH), 10.40/10.36 (s, CHO).

N-Trifluoroacetyl-3,5-dimethoxy-4-formylamphetamine, 16. To a solution of 26 mL (64.45 mmol, 3.0 eq) BuLi 2.5M in 60 mL THF anhydr. At −100° C. (cooling bath: acetone/THF approx. 4/1 mixture, adjusted with liquid N₂) under nitrogen was added dropwise (10 minutes) a solution of 8.0 g (21.61 mmol) 13 in 90 mL THF anhydr. In such a manner that a “precooling” on the flask's wall occurred (needle touched the flask's wall). After stirring for 5 minutes, 27.5 mL DMF anhydr. Were added within 5 minutes in a similar way. After completion of addition stirring was continued for 45 minutes and then the mixture was allowed to reach approx. −10° C. Next, the mixture was quenched by a quick addition of 150 mL aq. Saturated NH₄Cl solution and 150 mL of citric acid 10%. The mixture was extracted with EtOAc (2×150 mL), and the combined org. extracts were washed twice with water (2×150 mL) and with brine (1×150 mL), dried over Na₂SO₄ and concentrated in vacuo to get 6.64 g of a crude off-white solid. The residue was dissolved in 200 mL EtOAc and 20 mL MeOH. To this solution 18 g silica gel were added and the solvents were removed by rotary evaporation at 45° C. The residue was packed onto a silica gel dry flash column (h: 4 cm, d: 8 cm) preconditioned with hexane/EtOAc 3:1. It was eluted with the same eluent until all the unwanted by-product (N-trifluoroacetyl-3,5-dimethoxyamphetamine) was eluted, then it was changed to hexane/EtOAc 1:2; 1:3; 1:4 and finally pure EtOAc in order to elute the desired product. After pooling and evaporating the corresponding fractions there were obtained 3.33 g (48.3%) product 16 as a beige solid. ¹H-NMR (CDCl₃): 1.31 (d, MeCH), 2.89 (m, ArCH₂), 3.91 (s, 2MeO), 4.37 (m, CHNH), 6.19 (bs, NH), 6.40 (s, 2 arom. H), 10.47 (s, CHO).

### 3. Wittig Reactions

N-Trifluoroacetyl-3,5-dimethoxy-4-(2,2-dimethylvinyl)phenethylamine, 17a. To an ice-cooled suspension of 2.62 g (2.3 eq) isopropyltriphenylphosphonium iodide in 25 mL THF anhydr. was added 2.36 mL (2.2 eq) BuLi 2.5M in 25 mL THF anh. during 2 minutes (immediate coloration towards a deep orange). After stirring for 10 minutes at 0° C., a solution of 0.80 g (2.63 mmol) aldehyde 15 in 12 mL THF anhydr. Was added over 3 minutes whereby the reaction mixture did not discolor (and thus, some of the Wittig reagent still was intact, e.g., the excess, and was not “over”-consumed). After stirring for 3 hours at 0° C., the mixture was stirred overnight at ambient temperature. Next, 2 mL acetone were added and the mixture was concentrated in vacuo. The residue was partitioned between ethyl acetate and water (50 mL, each), and the organic layer was dried over Na₂SO₄ and concentrated in vacuo. The residue was redissolved in 7 mL DMSO and subjected to preparative HPLC purification (Dynamax SD1, 100 mL/min; UV detection at 210 nm; column: Macherey Nagel, reversed phase C18 Nucleodur Pyramid, 250×32, solvents: A: aqueous formic acid 0.05%, B: acetonitrile, gradient of 80% A and 20% B towards 5% A and 95% B). The collected fractions were roughly concentrated in vacuo to get rid of the acetonitrile. The residue was extracted with ethyl acetate; some brine helped to facilitate phase separation, and the organic layer was dried over Na₂SO₄ and concentrated in vacuo to get 0.741 g product as an orangish solid. The residue was dissolved in a minimal amount of DCM and added onto a 2 cm silica gel pad (d: 3 cm) preconditioned with ethyl acetate/hexane 1:1. Then it was eluted (approx. 150 mL) with ethyl acetate/hexane 1:1, and the eluate was concentrated in vacuo to get 710 mg (81.8%) product 17a as a beige solid. ¹H-NMR (CDCl₃) revelated an approx. 90:10 mixture of cis/trans amide mixture (isomers not assigned; only major product peak's shifts given): 1.57 (d, 3H, MeC═), 1.97 (d, 3H, MeC═), 2.89 (t, ArCH₂), 3.67 (q (superimposed txd), CH₂NH), 3.82 (s, 2MeO), 5.96 (m, ArCH═), 6.34 (bs, NH), 6.39 (s, 2 arom. H).

N-Trifluoroacetyl-4-(Z-buta-1,3-dienyl)-3,5-dimethoxyphenethylamine, 17b. It followed exactly the procedure described for the preparation of compound 17a, by using allyltriphenylphosphonium bromide as the Wittig salt. From 0.80 g (2.63 mmol) aldehyde 15 there were obtained 227.4 mg (26.3%) product 17b as a white solid. ¹H-NMR (CDCl₃) revelated an approx. 90:10 mixture of cis/trans amide mixture (isomers not assigned; only major product peak's shifts given). For assignment of E/Z isomerism it was followed the comprehensive work of Byrne and Gilheany (Byrne et al., 2012). Z-isomer (exclusively formed or isolated): 2.91 (t, ArCH₂), 3.67 (q (superimposed txd), CH₂NH), 3.82 (s, 2MeO), 5.11 (dxm, 1H, H₂C═), 5.28 (dxm, 1H, H₂C═), 6.21-6.43 (m, 3 vinylic H), 6.35 (bs, NH), 6.41 (s, 2 arom. H).

N-Trifluoroacetyl-3,5-dimethoxy-4-vinylamphetamine, 18a. It followed exactly the procedure described for the preparation of compound 17a, by using methyltriphenylphosphonium bromide as the Wittig salt. The crude product was directly purified by classical silica gel chromatography (hexane/EtOAc 3:1). From 0.80 g (2.51 mmol) aldehyde 16 there were obtained 600 mg (75.3%) product 18a as a white solid. ¹H-NMR (CDCl₃): 1.25 (d, MeCH), 2.82 (m, ArCH₂), 3.83 (s, 2MeO), 4.31 (m, CHNH), 5.43 (dxd, 1H, H₂C═), 6.04 (dxd, 1H, H₂C═), 6.11 (bs, NH), 6.34 (s, 2 arom. H), 6.92 (dxd, 1H, ArCH═).

N-Trifluoroacetyl-3,5-dimethoxy-4-(2,2-dimethylvinyl)amphetamine, 18b. It followed exactly the procedure described for the preparation of compound 17a, by using isopropyltriphenylphosphonium iodide as the Wittig salt. The crude product was directly purified by classical silica gel chromatography (hexane/EtOAc 3:1). From 0.80 g (2.51 mmol) aldehyde 16 there were obtained 580 mg (66.9%) product 18a as a white solid. ¹H-NMR (CDCl₃): 1.26 (d, MeCH), 1.54 (d, 3H, MeC═), 1.94 (d, 3H, MeC═), 2.84 (m, ArCH₂), 3.79 (s, 2MeO), 4.32 (m, CHNH), 5.94 (m, ArCH═), 6.12 (bd, NH), 6.34 (s, 2 arom. H).

E- and Z-isomer of N-trifluoroacetyl-4-(buta-1,3-dienyl)-3,5-dimethoxy-amphetamine, 18c and 18d. It followed exactly the procedure described for the preparation of compound 17a, by using allyltriphenylphosphonium bromide as the Wittig salt. The crude product was first purified by classical silica gel chromatography (hexane/EtOAc 3:1). Next, the pure E/Z-mixture 18c/18d was separated by preparative HPLC applying the same conditions as described under the preparation of 17a. From 0.80 g (2.51 mmol) aldehyde 16 there were obtained 130 mg (15.1%) E-isomer 18c and 360 mg (41.8%) Z-isomer 18d, each as a white solid. For assignment of E/Z isomerism it was followed the comprehensive work of Byrne and Gilheany Byrne and Gilheany (Byrne et al., 2012). ¹H-NMR (CDCl₃), E-isomer 18c: 1.27 (d, MeCH), 2.85 (m, ArCH₂), 3.87 (s, 2 MeO), 4.34 (m, CHNH), 5.12 (dxm, 1H, H₂C═), 5.30 (dxm, 1H, H₂C═), 6.11 (bd, NH), 6.36 (s, 2 arom. H), 6.53 (m, 1 vinylic H), 6.88 (m, 1 vinylic H), 7.24 (m, 1 vinylic H). Z-isomer 18d: 1.29 (d, MeCH), 2.87 (m, ArCH₂), 3.82 (s, 2MeO), 4.35 (m, CHNH), 5.11 (dxm, 1H, H₂C═), 5.27 (dxm, 1H, H₂C═), 6.14 (bd, NH), 6.21-6.42 (m, 3 vinylic H), 6.38 (s, 2 arom. H).

E- and Z-isomer of N-trifluoroacetyl-3,5-dimethoxy-4-(2-phenylvinyl)-amphetamine, 18e and 18f. It followed exactly the procedure described for the preparation of compound 17a, by using benzyltriphenylphosphonium chloride as the Wittig salt. The crude product was first purified by classical silica gel chromatography (hexane/EtOAc 3:1). Next, the pure E/Z-mixture 18e/18f (0.94 g) was separated by preparative HPLC applying the same conditions as described under the preparation of 17a. From 0.80 g (2.51 mmol) aldehyde 16 there were obtained 400 mg (40.6%) E-isomer 18e and 330 mg (33.4%) Z-isomer 18f, each as a white solid. For assignment of E/Z isomerism it was followed the comprehensive work of Byrne and Gilheany (Byrne et al., 2012). ¹H-NMR (CDCl₃), E-isomer 18e: 1.27 (d, MeCH), 2.84 (m, ArCH₂), 3.88 (s, 2MeO), 4.33 (m, CHNH), 6.12 (bd, NH), 6.38 (s, 2 arom. H), 7.18-7.59 (m, 5 arom. H plus 2 vinylic H). Z-isomer 18f: 1.28 (d, MeCH), 2.84 (d, ArCH₂), 3.58 (s, 2MeO), 4.33 (m, CHNH), 6.12 (bd, NH), 6.30 (s, 2 arom. H), 6.41 (d, 1 vinylic H), 6.71 (d, 1 vinylic H), 7.07-7.16 (m, 5 arom. H).

### 4. N-Trifluoroacetate Hydrolysis and Hydrochloride Salt Formation

3,5-Dimethoxy-4-(2,2-dimethylvinyl)phenethylamine hydrochloride (Desoxydimethylvinylscaline; DDMV), 19a. To a solution of 700 mg (2.11 mmol) 17a in 120 mL MeOH were added 34.5 mL aq. NaOH 5M under nitrogen. After stirring for 90 minutes, about ½ of the MeOH was roughly removed by using a rotary evaporator (<30° C.), and the mixture was diluted with 100 mL MTBE and washed with 3×30 mL water, dried over Na₂SO₄ and concentrated in vacuo to get 414 mg product (oil) as free base of 19a. This was dissolved in a few drops of isopropanol anhydr. And 20 mL diethyl ether anhydr. And carefully neutralized under stirring by adding HCl 2M anhydr. In diethyl ether (pH paper was used for check). The white precipitation was filtered off and rinsed with diethyl ether and dried under vacuum to get 426 mg (74.3%) product 19a as an off-white solid. ¹H-NMR (D₂O): 1.41 (d, 3H, MeC═), 1.83 (d, 3H, MeC═), 2.94 (t, ArCH₂), 3.24 (t, CH₂NH₃+), 3.75 (s, 2MeO), 5.80 (m, ArCH═), 6.62 (s, 2 arom. H).

4-(Z-Buta-1,3-dienyl)-3,5-dimethoxyphenethylamine hydrochloride (Z-Desoxybutadienylscaline; Z-DBD), 19b. It followed exactly the procedure (hydrolysis and hydrochloride salt formation) described for the preparation of compound 19a, by using 220 mg 17b and 11.5 mL aq. NaOH 5M in 40 mL MeOH. Yield: 137 mg (76.0%) product 19b as a pale-yellowish solid. ¹H-NMR (D₂O): 2.95 (t, ArCH₂), 3.25 (t, CH₂NH₃+), 3.75 (s, 2MeO), 5.13 (dxm, 1H, H₂C═), 5.30 (dxm, 1H, H₂C═), 6.15 (m, 2 vinylic H), 6.36 (m, 1 vinylic H), 6.65 (s, 2 arom. H).

3,5-Dimethoxy-4-vinylamphetamine hydrochloride (3C-DV), 20a. It followed exactly the procedure (hydrolysis and hydrochloride salt formation) described for the preparation of compound 19a, by using 600 mg 18a and 25 mL aq. NaOH 5M in 100 mL MeOH. Yield: 420 mg (86.2%) product 20a as a white solid. ¹H-NMR (D₂O): 1.22 (d, MeCH), 2.82 (d, ArCH₂), 3.58 (m, CHNH₃+), 3.76 (s, 2MeO), 5.41 (dxd, 1H, H₂C═), 5.87 (dxd, 1H, H₂C═), 6.55 (s, 2 arom. H), 6.74 (dxd, 1H, ArCH═).

3,5-Dimethoxy-4-(2,2-dimethylvinyl)amphetamine hydrochloride (3C-DDMV), 20b. It followed exactly the procedure (hydrolysis and hydrochloride salt formation) described for the preparation of compound 19a, by using 580 mg 18b and 25 mL aq. NaOH 5M in 100 mL MeOH. Yield: 460 mg (95.8%) product 20b as a white solid. ¹H-NMR (D₂O): 1.22 (d, MeCH), 1.39 (s, 3H, MeC═), 1.79 (s, 3H, MeC═), 2.84 (d, ArCH₂), 3.57 (m, CHNH₃+), 3.71 (s, 2MeO), 5.76 (s, ArCH═), 6.56 (s, 2 arom. H).

4-(E-Buta-1,3-dienyl)-3,5-dimethoxyamphetamine hydrochloride (E-3C-DBD), 20c. It was exactly followed the procedure (hydrolysis and hydrochloride salt formation) described for the preparation of compound 19a, by using 130 mg 18c and 5 mL aq. NaOH 5M in 20 mL MeOH. Yield: 60 mg (55.8%) product 20c as a yellowish-white solid. ¹H-NMR (D₂O): 1.22 (d, MeCH), 2.83 (d, ArCH₂), 3.57 (m, CHNH₃+), 3.78 (s, 2MeO), 5.10 (dxm, 1H, H₂C═), 5.26 (dxm, 1H, H₂C═), 6.47 (m, 1 vinylic H), 6.54 (s, 2 arom. H), 6.71 (d, 1 vinylic H), 7.07 (m, 1 vinylic H).

4-(Z-Buta-1,3-dienyl)-3,5-dimethoxyamphetamine hydrochloride (Z-3C-DBD), 20d. It followed exactly the procedure (hydrolysis and hydrochloride salt formation) described for the preparation of compound 19a, by using 360 mg 18d and 20 mL aq. NaOH 5M in 80 mL MeOH. Yield: 240 mg (80.8%) product 20d as a white solid. ¹H-NMR (D₂O): 1.23 (d, MeCH), 2.85 (dxd, ArCH₂), 3.58 (m, CHNH₃+), 3.71 (s, 2MeO), 5.10 (dxm, 1H, H₂C═), 5.27 (dxm, 1H, H₂C═), 6.11 (m, 2 vinylic H), 6.32 (t, 1 vinylic H), 6.58 (s, 2 arom. H).

3,5-Dimethoxy-4-(E-2-phenylvinyl)amphetamine hydrochloride (E-3C-DPV), 20d. It followed exactly the procedure (hydrolysis and hydrochloride salt formation) described for the preparation of compound 19a, by using 400 mg 18e and 20 mL aq. NaOH 5M in 80 mL MeOH. Yield: 185 mg (54.6%) product 20e as a white solid. ¹H-NMR (D₂O): 1.18 (d, MeCH), 2.77 (m, ArCH₂), 3.50 (m, CHNH₃+), 3.72 (s, 2MeO), 6.47 (s, 2 arom. H), 7.10-7.45 (m, 5 arom. H plus 2 vinylic H).

3,5-Dimethoxy-4-(Z-2-phenylvinyl)amphetamine hydrochloride (Z-3C-DPV), 20f. It followed exactly the procedure (hydrolysis and hydrochloride salt formation) described for the preparation of compound 19a, by using 330 mg 18f and 20 mL aq. NaOH 5M in 80 mL MeOH. Yield: 250 mg (89.3%) product 20f as an off-white solid. ¹H-NMR (D₂O): 1.22 (d, MeCH), 2.86 (m, ArCH₂), 3.53 (s, 2MeO), 3.56 (m, CHNH₃+), 6.32 (d, 1 vinylic H), 6.55 (s, 2 arom. H), 6.70 (d, 1 vinylic H), 7.01-7.17 (m, 5 arom. H).

### Examples—Preparation of the Desoxyscalines 22a-c Via Stille Reaction, N-Trifluoroacetate Hydrolysis and Conversion to the Hydrochloride Salts

### 1. Stille Reaction

N-Trifluoroacetyl-3,5-dimethoxy-4-vinylphenethylamine, 21a. This procedure has been adapted from (Saa et al., 1992). A mixture of 0.80 g (2.25 mmol) aryl halide 13, 355 mg (1.34 mmol) PPh₃, 177 mg (0.267 mmol) PdCl₂(PPh₃)₂ and 0.77 g (18.9 mmol) LiCl anhydr. in 20 mL DMF anhydr. was briefly degassed with N₂ using a balloon attached to a syringe/needle. Next, a crystal of BHT (3,5-di-tert-butyl-4-hydroxytoluene) and 0.787 mL (2.69 mmol; d: 1.085 g/mL) vinyltributylstannane was added and the mixture was heated to 120° C. After 1 hour and 9 hours there was added another equal amount of the stannane reagent. After heating for a total of 24 hours the mixture was cooled to ambient temperature, diluted with Et₂O/water (150 mL each), the layers were separated and the organic layer was washed once with water, dried over Na₂SO₄ and concentrated in vacuo. The residue that partially crystallized was dissolved in 7 mL DMSO and subjected to preparative HPLC purification (Macherey Nagel, reversed phase C18 Nucleodur Pyramid, 250×32, 5 μm, solvent: gradient of 80% A: aqueous formic acid 0.05% and 20% B: acetonitrile towards 5% A and 95% B). The collected fractions were roughly concentrated in vacuo to get rid of the acetonitrile. The residue was extracted with ethyl acetate; some brine helped to facilitate phase separation, and the organic layer was dried over Na₂SO₄ and concentrated in vacuo to get 0.35 g product as a beige solid. ¹H-NMR (CDCl₃) of this material indicated that still some impurities were contained. Thus, the residue was dissolved in a minimal amount of dichloromethane and placed onto a 2 cm silica gel pad (d: 3 cm) preconditioned with ethyl acetate/hexane 1:1. Then it was eluted (approx. 150 mL) with ethyl acetate/hexane 1:1, and the eluate was concentrated in vacuo to get 340.1 mg (50.0%) product as a white solid. ¹H-NMR (CDCl₃) revelated an approx. 95:5 mixture of cis/trans amide mixture (isomers not assigned; only major product peak's shifts given): 2.88 (t, ArCH₂), 3.65 (q (superimposed txd), CH₂NH), 3.86 (s, 2MeO), 5.49 (dxd, 1H, H₂C═), 6.07 (dxd, 1H, H₂C═), 6.32 (bs, NH), 6.39 (s, 2 arom. H), 6.95 (dxd, 1H, ArCH═).

N-Trifluoroacetyl-3,5-dimethoxy-4-ethynylphenethylamine, 21b. It was exactly followed the procedure described for the preparation of compound 21a, by using ethynyltributylstannane (d: 1.089 g/mL) as the alkylating reagent. From 0.80 g aryl halide 13 were obtained 91.6 mg (13.3%) product as a beige solid. ¹H-NMR (CDCl₃): 2.92 (t, ArCH₂), 3.58 (s, 1H, HC≡), 3.66 (q (superimposed txd), CH₂NH), 3.92 (s, 2MeO), 6.33 (bs, NH), 6.39 (s, 2 arom. H).

N-Trifluoroacetyl-4-allyl-3,5-dimethoxyphenethylamine, 21c. It was exactly followed the procedure described for the preparation of compound 21a, by using allyltributylstannane (d: 1.07 g/mL) as the alkylating reagent. From 1.20 g aryl halide 13 were obtained 459 mg (42.9%) product as a beige solid. ¹H-NMR (CDCl₃) revelated an approx. 90:10 mixture of cis/trans amide mixture (isomers not assigned; only major product peak's shifts given): 2.88 (t, ArCH₂), 3.39 (dm, ArCH₂CH═CH₂), 3.65 (q (superimposed txd), CH₂NH), 3.83 (s, 2MeO), 4.98 (m, 2H, H₂C═C), 5.95 (m, H₂C═CH), 6.32 (bs, NH), 6.38 (s, 2 arom. H).

### 2. N-Trifluoroacetate Hydrolysis and Hydrochloride Salt Formation

3,5-Dimethoxy-4-vinylphenethylamine hydrochloride (Desoxvinylscaline; DV), 22a. It followed exactly the procedure (hydrolysis and hydrochloride salt formation) described for the preparation of compound 19a, by using 330 mg 21a and 18 mL aq. NaOH 5M in 60 mL MeOH. Yield: 128 mg (48.3%) product 22a as a white solid. ¹H-NMR (D₂O): 2.92 (t, ArCH₂), 3.23 (t, CH₂NH₃+), 3.80 (s, 2MeO), 5.44 (dxd, 1H, H₂C═), 5.90 (dxd, 1H, H₂C═), 6.61 (s, 2 arom. H), 6.78 (dxd, 1H, ArCH═).

3,5-Dimethoxy-4-ethynylphenethylamine hydrochloride (Desoxyethynylscaline; DYN), 22b. It followed exactly the procedure (hydrolysis and hydrochloride salt formation) described for the preparation of compound 19a, by using 91.6 mg 21b and 5 mL aq. NaOH 5M in 17 mL MeOH. Yield: 14.1 mg (19.2%) product 22b as an off-white solid. ¹H-NMR (D₂O): 2.95 (t, ArCH₂), 3.24 (t, CH₂NH₃+), 3.84 (s, 2MeO), 3.89 (s, 1H, HCE), 6.62 (s, 2 arom. H).

4-Allyl-3,5-dimethoxyphenethylamine hydrochloride (Desoxyallylscaline; DAL), 22c. It followed exactly the procedure (hydrolysis and hydrochloride salt formation) described for the preparation of compound 19a, by using 440 mg 21c and 23 mL aq. NaOH 5M in 80 mL MeOH. Yield: 315 mg (88.1%) product 22c as a white solid. ¹H-NMR (D₂O): 2.93 (t, ArCH₂), 3.23 (t, CH₂NH₃+), 3.29 (dxt, ArCH₂CH═CH₂), 3.77 (s, 2MeO), 4.80 (dxm, 1H, H₂C═C), 4.90 (dxm, 1H, H₂C═C), 5.93 (m, H₂C═CH), 6.62 (s, 2 arom. H).

### REFERENCES

1. Azzena U, Cossu S, Denurra T, Melloni G, & Piroddi A M (1990). Regioselective Reductive Alkylation of 3,4,5-Trimethoxybenzaldehyde Dimethylacetal: A New Synthesis of 4-Alkyl-3,5-dimethoxybenzaldehydes and 2,5-Dialkyl-1,3-dimethoxybenzenes. Synthesis 4: 313-314.
2. Benington F, Morin R D, & Clark L C (1960). Synthesis of some vivinal trimethoxyphenyl derivatives of heterocyclic nitogen bases. J Organ Chem 25: 1916-1919.
3. Bogenschutz MP (2013). Studying the effects of classic hallucinogens in the treatment of alcoholism: rationale, methodology, and current research with psilocybin. Curr Drug Abuse Rev 6: 17-29.
4. Bogenschutz M P, Forcehimes A A, Pommy J A, Wilcox C E, Barbosa P C, & Strassman R J (2015). Psilocybin-assisted treatment for alcohol dependence: a proof-of-concept study. J Psychopharmacol 29: 289-299.
5. Byrne P A, & Gilheany D G (2012). Unequivocal experimental evidence for a unified lithium salt-free Wittig reaction mechanism for all phosphonium Ylide types: reactions with beta-heteroatom-substituted aldehydes are consistently selective for cis-oxaphosphetane-derived products. J Am Chem Soc 134: 9225-9239.
6. Carhart-Harris R, Giribaldi B, Watts R, Baker-Jones M, Murphy-Beiner A, Murphy R, et al. (2021). Trial of psilocybin versus escitalopram for depression. N Engl J Med 384: 1402-1411.
7. Carhart-Harris R L, Bolstridge M, Rucker J, Day C M, Erritzoe D, Kaelen M, et al. (2016a). Psilocybin with psychological support for treatment-resistant depression: an open-label feasibility study. Lancet Psychiatry 3: 619-627.
8. Carhart-Harris R L, Kaelen M, Bolstridge M, Williams T M, Williams L T, Underwood R, et al. (2016b). The paradoxical psychological effects of lysergic acid diethylamide (LSD). Psychol Med 46: 1379-1390.
9. Charalampous W, Kinross-Wright (1966). Metabolic Fate of Mescaline in Man. Psychopharmacologia 9: 48-63.
10. Comins D L, & Brown J D (1984). Ortho metalation directed by alpha-amino alkoxides. J Org Chem 49: 1078-1083.
11. Davis A K, Barrett F S, May D G, Cosimano M P, Sepeda N D, Johnson M W, et al. (2021). Effects of psilocybin-assisted therapy on major depressive disorder: a randomized clinical trial. JAMA Psychiatry 78: 481-489.
12. de Araujo D B Acute, lasting and antidepressant effects of Ayahuasca.
13. Dolder P C, Schmid Y, Mueller F, Borgwardt S, & Liechti M E (2016). LSD acutely impairs fear recognition and enhances emotional empathy and sociality. Neuropsychopharmacology 41: 2638-2646.
14. Dolder P C, Schmid Y, Steuer A E, Kraemer T, Rentsch K M, Hammann F, et al. (2017). Pharmacokinetics and pharmacodynamics of lysergic acid diethylamide in healthy subjects. Clin Pharmacokinetics 56: 1219-1230.
15. Dominguez-Clave E, Soler J, Elices M, Pascual J C, Alvarez E, de la Fuente Revenga M, et al. (2016). Ayahuasca: Pharmacology, neuroscience and therapeutic potential. Brain Res Bull 126: 89-101.
16. Dos Santos R G, Osorio F L, Crippa J A, Riba J, Zuardi A W, & Hallak J E (2016). Antidepressive, anxiolytic, and antiaddictive effects of ayahuasca, psilocybin and lysergic acid diethylamide (LSD): a systematic review of clinical trials published in the last 25 years. Ther Adv Psychopharmacol 6: 193-213.
17. Garcia-Romeu A, Davis A K, Erowid F, Erowid E, Griffiths R R, & Johnson M W (2019). Cessation and reduction in alcohol consumption and misuse after psychedelic use. J Psychopharmacol: 269881119845793.
18. Garcia-Romeu A, Griffiths R R, & Johnson M W (2015). Psilocybin-occasioned mystical experiences in the treatment of tobacco addiction. Curr Drug Abuse Rev 7: 157-164.
19. Gasser P, Holstein D, Michel Y, Doblin R, Yazar-Klosinski B, Passie T, et al. (2014). Safety and efficacy of lysergic acid diethylamide-assisted psychotherapy for anxiety associated with life-threatening diseases. J Nery Ment Dis 202: 513-520.
20. Gasser P, Kirchner K, & Passie T (2015). LSD-assisted psychotherapy for anxiety associated with a life-threatening disease: a qualitative study of acute and sustained subjective effects. J Psychopharmacol 29: 57-68.
21. Griffiths R, Richards W, Johnson M, McCann U, & Jesse R (2008). Mystical-type experiences occasioned by psilocybin mediate the attribution of personal meaning and spiritual significance 14 months later. J Psychopharmacol 22: 621-632.
22. Griffiths R R, Johnson M W, Carducci M A, Umbricht A, Richards W A, Richards B D, et al. (2016). Psilocybin produces substantial and sustained decreases in depression and anxiety in patients with life-threatening cancer: a randomized double-blind trial. J Psychopharmacol 30: 1181-1197.
23. Grob C S, Danforth A L, Chopra G S, Hagerty M, McKay C R, Halberstadt A L, et al. (2011). Pilot study of psilocybin treatment for anxiety in patients with advanced-stage cancer. Arch Gen Psychiatry 68: 71-78.
24. Holze F, Duthaler U, Vizeli P, Muller F, Borgwardt S, & Liechti M E (2019). Pharmacokinetics and subjective effects of a novel oral LSD formulation in healthy subjects. Br J Clin Pharmacol 85: 1474-1483.
25. Holze F, Vizeli P, Ley L, Muller F, Dolder P, Stocker M, et al. (2021). Acute dose-dependent effects of lysergic acid diethylamide in a double-blind placebo-controlled study in healthy subjects. Neuropsychopharmacology 46: 537-544.
26. Hysek C M, Simmler L D, Nicola V, Vischer N, Donzelli M, Krähenbühl S, et al. (2012). Duloxetine inhibits effects of MDMA (“ecstasy”) in vitro and in humans in a randomized placebo-controlled laboratory study. PLoS One 7: e36476.
27. Ichikawa J, & Meltzer H Y (2000). The effect of serotonin (1A) receptor agonism on antipsychotic drug-induced dopamine release in rat striatum and nucleus accumbens. Brain Res 858: 252-263.
28. Johnson M W, Garcia-Romeu A, Cosimano M P, & Griffiths R R (2014). Pilot study of the 5-HT2A R agonist psilocybin in the treatment of tobacco addiction. J Psychopharmacol 28: 983-992.
29. Johnson M W, Garcia-Romeu A, & Griffiths R R (2016). Long-term follow-up of psilocybin-facilitated smoking cessation. Am J Drug Alcohol Abuse 43: 55-60.
30. Krebs T S, & Johansen P O (2012). Lysergic acid diethylamide (LSD) for alcoholism: meta-analysis of randomized controlled trials. J Psychopharmacol 26: 994-1002.
31. Liechti M E (2017). Modern clinical research on LSD. Neuropsychopharmacology 42: 2114-2127.
32. Luethi D, & Liechti M E (2018). Monoamine Transporter and Receptor Interaction Profiles in Vitro Predict Reported Human Doses of Novel Psychoactive Stimulants and Psychedelics. Int J Neuropsychopharmacol 21: 926-931.
33. Madsen M K, Fisher P M, Burmester D, Dyssegaard A, Stenbaek D S, Kristiansen S, et al. (2019). Correction: Psychedelic effects of psilocybin correlate with serotonin 2A receptor occupancy and plasma psilocin levels. Neuropsychopharmacology 44: 1328-1334.
34. Marona-Lewicka D, Thisted R A, & Nichols D E (2005). Distinct temporal phases in the behavioral pharmacology of LSD: dopamine D2 receptor-mediated effects in the rat and implications for psychosis. Psychopharmacology (Berl) 180: 427-435.
35. Palhano-Fontes F, Barreto D, Onias H, Andrade K C, Novaes M M, Pessoa J A, et al. (2019). Rapid antidepressant effects of the psychedelic ayahuasca in treatment-resistant depression: a randomized placebo-controlled trial. Psychol Med 49: 655-663.
36. Passie T, Halpern J H, Stichtenoth D O, Emrich H M, & Hintzen A (2008). The pharmacology of lysergic acid diethylamide: a review. CNS Neurosci Ther 14: 295-314.
37. Rickli A, Moning O D, Hoener M C, & Liechti M E (2016). Receptor interaction profiles of novel psychoactive tryptamines compared with classic hallucinogens. Eur Neuropsychopharmacol 26: 1327-1337.
38. Roseman L, Nutt D J, & Carhart-Harris R L (2017). Quality of acute psychedelic experience predicts therapeutic efficacy of psilocybin for treatment-resistant depression. Front Pharmacol 8: 974.
39. Ross S, Bossis A, Guss J, Agin-Liebes G, Malone T, Cohen B, et al. (2016). Rapid and sustained symptom reduction following psilocybin treatment for anxiety and depression in patients with life-threatening cancer: a randomized controlled trial. J Psychopharmacol 30: 1165-1180.
40. Saa J M, Martorell G, & Garcia-Raso A (1992). Palladium-catalyzed cross-coupling reactions of highly hindered, electron-rich phenol triflates and organostannanes. J Org Chem 57: 678-685.
41. Sanches R F, de Lima Osorio F, Dos Santos R G, Macedo L R, Maia-de-Oliveira J P, Wichert-Ana L, et al. (2016). Antidepressant Effects of a Single Dose of Ayahuasca in Patients With Recurrent Depression: A SPECT Study. J Clin Psychopharmacol 36: 77-81.
42. Schmid Y, Enzler F, Gasser P, Grouzmann E, Preller K H, Vollenweider F X, et al. (2015). Acute effects of lysergic acid diethylamide in healthy subjects. Biol Psychiatry 78: 544-553.
43. Schmid Y, Gasser P, Oehen P, & Liechti M E (2021). Acute subjective effects in LSD- and MDMA-assisted psychotherapy. J Psychopharmacol 35: 362-374.
44. Schmid Y, & Liechti M E (2018). Long-lasting subjective effects of LSD in normal subjects. Psychopharmacology (Berl) 235: 535-545.
45. Shulgin A, & Shulgin A (1991) PiHKAL: a chemical love story. 1 edn.
46. Trachsel D (2002). Synthese von neuen (Phenylalkyl)aminen zur Untersuchung von Struktur-Aktivitatsbeziehungen, Mitteilung 1: Mescalin Derivative. Helvetica Chimica Acta 85: 3019-3026.
47. Trachsel D, Lehmann D, & Enzensperger C (2013) Phenethylemine: Von der Struktur zur Funktion. 1 edn.: Solothurn: Nachtschatten Verlag.
48. Vollenweider F X, Vontobel P, Hell D, & Leenders K L (1999). 5-HT modulation of dopamine release in basal ganglia in psilocybin-induced psychosis in man—a PET study with [11C]raclopride. Neuropsychopharmacology 20: 424-433.
49. Zheng J, Cai J, Lin J H, Guo Y, & Xiao J C (2013). Synthesis and decarboxylative Wittig reaction of difluoromethylene phosphobetaine. Chem Commun (Camb) 49: 7513-7515.

What is claimed is:

1. A composition comprising a compound represented by FIG. 1,
characterized in that Rₐₗₚₕₐ₁ and, independently and in any combination, Rₐₗₚₕₐ₂ is chosen from the group consisting of hydrogen, deuteron, methyl, ethyl, deuterated methyl (D₁-D₃), or deuterated ethyl (D₁-D₅), and further characterized in that R′ is one of the following substituents:
C₁-C₅ branched or unbranched alkyl with the alkyl optionally substituted with F₁-F₁₁ fluorine and/or D₁-D₁₁ deuteron substituents up to a fully fluorinated and/or deuterated alkyl,
C₃-C₆ cycloalkyl optionally and independently substituted with one or more substituents chosen from the group consisting of F₁-F₁₅ fluorine, D₁-D₁₅ deuteron, C₁-C₂ alkyl, and combinations thereof,
(C₃-C₆ cycloalkyl)-C₁-C₂ branched or unbranched alkyl optionally substituted with one or more substituents chosen from the group consisting of F₁-F₁₅ fluorine, D₁-D₁₅ deuteron, C₁-C₂ alkyl, and combinations thereof,
C₂-C₅ branched or unbranched alkenyl with E or Z or cis or trans double bond configuration, where any of the carbons of the branched or unbranched alkenyl substituent is substituted with a substituent chosen from the group consisting of C₁-C₂ alkyl, F₁-F₁₃ fluorine, D₁-D₁₃ deuteron, C₂ alkenyl, aryl or heteroaryl bearing zero up to any number of ether, thioether, halogen, alkyl, fluorinated alkyl, alkenyl, alkynyl or nitrogen-containing substituents, and combinations thereof,
C₂-C₅ branched or unbranched alkynyl where any of the carbons of the branched or unbranched alkynyl substituent is substituted with a substituent chosen from the group consisting of one or more C₁-C₂ alkyl, F₁-F₁₁ fluorine, D₁-D₁₁ deuteron, C₂ alkenyl, aryl or heteroaryl bearing zero up to any number of ether, thioether, halogen, alkyl, fluorinated alkyl, alkenyl, alkynyl or nitrogen-containing substituents, and combinations thereof,
any halogen or
a nitrogen-containing substituent of CN or NO₂.

2. The composition of claim 1, wherein said compound is a free base.

3. The composition of claim 1, wherein said compound is a salt thereof.

4. The composition of claim 3, wherein said compound is a hydrochloride salt thereof.

5. The composition of claim 4, wherein said compound is a pharmacologically acceptable acid addition salt thereof chosen from the group consisting of sulfate, pyrosulfate, bisulfate, sulfite, bisulfite, phosphate, monohydrogen-phosphate, dihydrogenphosphate, metaphosphate, pyro-phosphate, chloride, bromide, iodide, formate, acetate, propionate, decanoate, caprylate, acrylate, isobutyrate, caproate, heptanoate, oxalate, malonate, succinate, suberate, sebacate, fumarate, maleate, benzoate, phthalate, sulfonate, phenylacetate, citrate, lactate, glycollate, tartrate, methanesulfonate, propanesulfonate, and mandelate.

6. The composition of claim 1, wherein said compound is chosen from the group consisting of a racemate, a single enantiomer, a diastereomer, and a mixture of enantiomers or diastereomers in any ratio, a single and a mixture E or Z configurational isomer in any ratio, a single and a mixture cis or trans configurational isomer in any ratio, and any combination thereof.

7. A method of changing neurotransmission, including the steps of:
administering a pharmaceutically effective amount of composition to a mammal of a compound represented by FIG. 1, which is characterized in that Rₐₗₚₕₐ₁ and, independently and in any combination, Rₐₗₚₕₐ₂ is chosen from the group consisting of hydrogen, deuteron, methyl, ethyl, deuterated methyl (D₁-D₃), or deuterated ethyl (D₁-D₅), and further characterized in that R′ is one of the following substituents:
C₁-C₅ branched or unbranched alkyl with the alkyl optionally substituted with F₁-F₁₁ fluorine and/or D₁-D₁₁ deuteron substituents up to a fully fluorinated and/or deuterated alkyl,
C₃-C₆ cycloalkyl optionally and independently substituted with one or more substituents chosen from the group consisting of F₁-F₁₅ fluorine, D₁-D₁₅ deuteron, C₁-C₂ alkyl, and combinations thereof,
(C₃-C₆ cycloalkyl)-C₁-C₂ branched or unbranched alkyl optionally substituted with one or more substituents chosen from the group consisting of F₁-F₁₅ fluorine, D₁-D₁₅ deuteron, C₁-C₂ alkyl, and combinations thereof,
C₂-C₅ branched or unbranched alkenyl with E or Z or cis or trans double bond configuration, where any of the carbons of the branched or unbranched alkenyl substituent is substituted with a substituent chosen from the group consisting of C₁-C₂ alkyl, F₁-F₁₃ fluorine, D₁-D₁₃ deuteron, C₂ alkenyl, aryl or heteroaryl bearing zero up to any number of ether, thioether, halogen, alkyl, fluorinated alkyl, alkenyl, alkynyl or nitrogen-containing substituents, and combinations thereof,
C₂-C₅ branched or unbranched alkynyl where any of the carbons of the branched or unbranched alkynyl substituent is substituted with a substituent chosen from the group consisting of one or more C₁-C₂ alkyl, F₁-F₁₁ fluorine, D₁-D₁₁ deuteron, C₂ alkenyl, aryl or heteroaryl bearing zero up to any number of ether, thioether, halogen, alkyl, fluorinated alkyl, alkenyl, alkynyl or nitrogen-containing substituents, and combinations thereof,
any halogen or
a nitrogen-containing substituent of CN or NO₂;
increasing serotonin 5-HT2A and 5-HT2C receptor interaction in the mammal; and
inducing psychoactive effects.

8. The method of claim 7, wherein the compound is chosen from the group consisting of a racemate, a single enantiomer, a diastereomer, a mixture of enantiomers or diastereomers in any ratio, a single and a mixture E or Z configurational isomer in any ratio, a single and a mixture cis or trans configurational isomer in any ratio, and any combination thereof.

9. The method of claim 7, wherein the psychoactive effects include psychedelic or empathogenic effects having intensity, effect quality, or duration of effect in a mammal in comparison to that of mescaline.

10. The method of claim 7, wherein the compound is administered to mammals for substance-assisted psychotherapy.

11. The method of claim 7, wherein the compound is administered to allow for changing dose potency in comparison to mescaline.

12. The method of claim 7, wherein the compound is administered to allow for tailoring and treatment individualization to the mammal's therapeutic need.

13. The method of claim 10, wherein the mammal is a human.

14. A method of treating a patient having adverse reactions to psychedelics, including the steps of:
administering a desoxyscaline derivative to the patient; and
avoiding adverse effects present with psychedelics.

15. The method of claim 14, wherein the adverse effects are chosen from the group consisting of anxiety, cardio-stimulant effects, thermogenesis, adverse effects, nausea, and combinations thereof.

16. The method of claim 15, further including the step of providing more positive effects than other psychedelics.

17. The method of claim 16, wherein the positive effects are chosen from the group consisting of more overall positive effects, more or less perceptual effects, more emotional effects, and combinations thereof.

18. The method of claim 14, further including the step of providing a shorter duration of action of the desoxyscaline derivative than with other psychedelics.

19. The method of claim 14, wherein the desoxyscaline derivative is further defined as a compound represented by FIG. 1, which is characterized in that Rₐₗₚₕₐ₁ and, independently and in any combination, Rₐₗₚₕₐ₂ is chosen from the group consisting of hydrogen, deuteron, methyl, ethyl, deuterated methyl (D₁-D₃), or deuterated ethyl (D₁-D₅), and further characterized in that R′ is one of the following substituents:
C₁-C₅ branched or unbranched alkyl with the alkyl optionally substituted with F₁-F₁₁ fluorine and/or D₁-D₁₁ deuteron substituents up to a fully fluorinated and/or deuterated alkyl,
C₃-C₆ cycloalkyl optionally and independently substituted with one or more substituents chosen from the group consisting of F₁-F₁₅ fluorine, D₁-D₁₅ deuteron, C₁-C₂ alkyl, and combinations thereof,
(C₃-C₆ cycloalkyl)-C₁-C₂ branched or unbranched alkyl optionally substituted with one or more substituents chosen from the group consisting of F₁-F₁₅ fluorine, D₁-D₁₅ deuteron, C₁-C₂ alkyl, and combinations thereof,
C₂-C₅ branched or unbranched alkenyl with E or Z or cis or trans double bond configuration, where any of the carbons of the branched or unbranched alkenyl substituent is substituted with a substituent chosen from the group consisting of C₁-C₂ alkyl, F₁-F₁₃ fluorine, D₁-D₁₃ deuteron, C₂ alkenyl, aryl or heteroaryl bearing zero up to any number of ether, thioether, halogen, alkyl, fluorinated alkyl, alkenyl, alkynyl or nitrogen-containing substituents, and combinations thereof,
C₂-C₅ branched or unbranched alkynyl where any of the carbons of the branched or unbranched alkynyl substituent is substituted with a substituent chosen from the group consisting of one or more C₁-C₂ alkyl, F₁-F₁₁ fluorine, D₁-D₁₁ deuteron, C₂ alkenyl, aryl or heteroaryl bearing zero up to any number of ether, thioether, halogen, alkyl, fluorinated alkyl, alkenyl, alkynyl or nitrogen-containing substituents, and combinations thereof,
any halogen or,
a nitrogen-containing substituent of CN or NO₂.

20. A method of changing neurotransmission of an individual, including the steps of:
administering a desoxyscaline derivative; and
changing neurotransmission in the individual.

21. The method of claim 20, wherein the desoxyscaline derivative is further defined as a compound represented by FIG. 1, which is characterized in that Rₐₗₚₕₐ₁ and, independently and in any combination, Rₐₗₚₕₐ₂ is chosen from the group consisting of hydrogen, deuteron, methyl, ethyl, deuterated methyl (D₁-D₃), or deuterated ethyl (D₁-D₅), and further characterized in that R′ is one of the following substituents:
C₁-C₅ branched or unbranched alkyl with the alkyl optionally substituted with F₁-F₁₁ fluorine and/or D₁-D₁₁ deuteron substituents up to a fully fluorinated and/or deuterated alkyl,
C₃-C₆ cycloalkyl optionally and independently substituted with one or more substituents chosen from the group consisting of F₁-F₁₅ fluorine, D₁-D₁₅ deuteron, C₁-C₂ alkyl, and combinations thereof,
(C₃-C₆ cycloalkyl)-C₁-C₂ branched or unbranched alkyl optionally substituted with one or more substituents chosen from the group consisting of F₁-F₁₅ fluorine, D₁-D₁₅ deuteron, C₁-C₂ alkyl, and combinations thereof,
C₂-C₅ branched or unbranched alkenyl with E or Z or cis or trans double bond configuration, where any of the carbons of the branched or unbranched alkenyl substituent is substituted with a substituent chosen from the group consisting of C₁-C₂ alkyl, F₁-F₁₃ fluorine, D₁-D₁₃ deuteron, C₂ alkenyl, aryl or heteroaryl bearing zero up to any number of ether, thioether, halogen, alkyl, fluorinated alkyl, alkenyl, alkynyl or nitrogen-containing substituents, and combinations thereof,
C₂-C₅ branched or unbranched alkynyl where any of the carbons of the branched or unbranched alkynyl substituent is substituted with a substituent chosen from the group consisting of one or more C₁-C₂ alkyl, F₁-F₁₁ fluorine, D₁-D₁₁ deuteron, C₂ alkenyl, aryl or heteroaryl bearing zero up to any number of ether, thioether, halogen, alkyl, fluorinated alkyl, alkenyl, alkynyl or nitrogen-containing substituents, and combinations thereof,
any halogen or,
a nitrogen-containing substituent of CN or NO₂.
